SEQUENCE LISTING (1 sequence)

SEQ ID NO: 1 - DNA, 21 bp, Artificial Sequence
  construct
cgagatgcttaagtgtgacaa

(19) United States
(12) Patent Application Publication
(10) Pub. No.: US 2013/0244950 A1
(43) Pub. Date: Sep. 19, 2013

(54) COMBINATION THERAPY FOR CHEMORESISTANT CANCERS

(71) Applicant: CELGENE CORPORATION, Summit, NJ (US)

(72) Inventors: John A. Copland, III, Ponte Verde Breach, FL (US); Han Win Tun, Jacksonville, FL (US); Simon James Cooper, Jacksonville, FL (US); Laura Ann Marlow, Jacksonville, FL (US)

(73) Assignee: Celgene Corporation, Summit, NJ (US)

(21) Appl. No.: 13/627,848

(22) Filed: Sep. 26, 2012

Related U.S. Application Data
(60) Provisional application No. 61/539,452, filed on Sep. 26, 2011.

(51) Int. Cl. A61K 38/00; C12Q 1/68; G01N 33/574; A61K 31/706

(52) U.S. Cl. CPC A61K 38/005; A61K 31/706; C12Q 1/6886; G01N 33/57415; G01N 33/57438
USPC 514/19.9; 435/6.12; 435/7.92; 435/7.4

(57) ABSTRACT

Methods of treating, preventing or managing triple negative breast cancer (TNBC) or clear cell renal cell carcinoma (ccRCC) are disclosed. The methods encompass the administration of an HDAC inhibitor romidepsin in combination with a cytidine analog. Pharmaceutical compositions and single unit dosage forms suitable for use in the methods provided herein are also disclosed.

### CROSS REFERENCE TO RELATED APPLICATIONS

This application claims the benefit of priority to U.S. Provisional Patent Application Ser. No. 61/539,452 filed Sep. 26, 2011, the disclosure of which is incorporated by reference herein in its entirety.

### FIELD

Provided are methods for treating chemoresistant cancers using a combination of a histone deacetylase (HDAC) inhibitor and a DNA methyltransferase inhibitor. In one embodiment, the HDAC inhibitor is romidepsin. In another embodiment, the DNA methyltransferase inhibitor is azacitidine or decitabine. In yet another embodiment, the chemoresistant cancer is triple negative breast cancer (TNBC) or clear cell renal cell carcinoma (ccRCC).

### BACKGROUND

Renal cell carcinoma (RCC) is the third most prevalent urological cancer, and is the 10th most common cause of cancer death in men and the 9th most common cause in women (van Spronsen et al. Crit Rev Oncol Hematol 55:177-91, 2005). Clear cell renal cell carcinoma (ccRCC) is the largest subtype of RCC and accounts for approximately 80% of all renal cancers.

Breast cancer is the most common cancer in women with triple negative breast cancer (TNBC) accounting for approximately 15% of newly diagnosed breast cancers. TNBCs are associated with poor prognosis, a higher mitotic index and younger age (Kreike et al. Breast Cancer Res.; 9:R65, 2007).

In ccRCC and breast cancer, early diagnosis and treatment dramatically increase median survival rates. Both diseases, when metastatic, are mostly aggressive and drug resistant. Development of metastatic disease in ccRCC patients reduces the 5 year survival rate to less than 10% (Pantuck et al., J Urol 166:1611-23, 2001) and in TNBC reduces the survival rate to around 18 months in the majority of patients (Berrada et al., Ann Oncol 21 Suppl 7:vii30-vii5, 2010).

Cancer is a multistep process facilitated by the accumulation of genetic abnormalities resulting in genomic instability and the mutation of tumor suppressor and oncogenic genes. Furthermore, epigenetic changes in cancer lead to modulations of gene expression through mechanisms of DNA methylation and histone deacetylation. The hypermethylation of cytosines in areas of rich CpG islands, and deacetylation of histones, which facilitate a tighter formation of chromatin, both contribute to the inappropriate silencing of gene expression. HDAC inhibitors such as trichostatin A (TSA) and romidepsin and demethyltransferase inhibitors such as decitabine (DAC; 5-aza-2′-deoxycytodine) and 5-azacitidine (VIDAZA®) are capable of reversing these epigenetic events and suppressing the cancer phenotype.

The histone deacetylase inhibitor romidepsin exhibits antitumor properties in human cell lines both in vitro and in vivo (Ueda et al., Biosci Biotechnol Biochem 58:1579-83, 1994). Numerous studies have identified that romidepsin treatment of tumor cells leads to the inhibition of angiogenesis and cell growth, while inducing apoptosis, cell death and cell differentiation (Jung M., Curr Med Chem 8:1505-11, 2001; Zhu et al., Cancer Res 61:1327-33, 2001; Sandor et al., Clin Cancer Res 8:718-28, 2002; Konstantinopoulos et al., Cancer Chemother Pharmacol 58:711-5, 2006; Liu et al., Mol Cancer Ther 7:1751-61, 2008). In 2009, romidepsin was approved by the FDA for the treatment of cutaneous T-cell lymphoma and peripheral T-cell lymphoma.

DNA methyltransferase inhibitors are analogues of cytosine that are incorporated into the DNA during replication before covalently linking with DNA methyltransferases (DNMTs), thus leading to global loss of gene methylation (Christman J. K., Oncogene 21:5483-95, 2002). Treatment of cancer cell models with decitabine leads to suppression of growth and apoptosis through re-expression of silenced genes (Bender et al., Cancer Res 58:95-101, 1998; Herman et al., N Engl J Med 349:2042-54, 2003) and through the activation of p53 and p21^{Waf1/Cip1} (Zhu et al., J Biol Chem 279:15161-6, 2004). Recent studies have identified that decitabine causes G2 arrest, reduces clonogenic survival, and inhibits growth in cells while causing DNA fragmentation and activating the ATM and ATR DNA repair pathways (Palii et al., Mol Cell Biol 28:752-71, 2008). In 2006 decitabine was approved by the FDA for the treatment of myelodysplastic syndromes.

Constitutive activation of the Wnt signaling pathway as a mechanism for cancer development was first identified in colon cancer (Korinek et al., Science 275:1784-7, 1997). The binding of secreted Wnt family members to Frizzled receptor complexes on the cell surface leads to activation of downstream gene targets through either the canonical/β-catenin pathway or one of the non-canonical/β-catenin independent pathways (Widelitz R., Growth Factors; 23:111-6, 2005). Which of these pathways are activated is governed by the composition of the Wnt/Frizzled complex. The canonical Wnt signaling pathway influences genes associated with cell proliferation, survival and invasion (Gumz et al., Clin Cancer Res 13:4740-9, 2007), whilst the non-canonical pathways activates those involved in cell adhesion, migration and cytoskeletal reorganization (Komiya et al., Organogenesis 4:68-75, 2008). sFRP1, secreted frizzled related protein 1, functions as a negative regulator of Wnt signaling by sequestering Wnt proteins and by heterodimerizing with Frizzled to form non-functional receptor complexes. However in colorectal (Suzuki et al., Nat Genet 36:417-22, 2004), ovarian (Takada et al., Cancer Sci 95:741-4, 2004); lung (Fukui et al., Oncogene 24:6323-7, 2005); hepatocellular (Shih et al., Int J Cancer 121:1028-35, 2007); kidney (Gumz et al., supra) and breast cancer (Lo et al., Cancer Biol Ther 5:281-6, 2006) hypermethylation of the sFRP1 promoter and subsequent loss of expression has been identified allowing aberrant Wnt signaling through the canonical or non-canonical pathways.

Strategies to re-express epigenetically silenced genes are an attractive chemotherapy option in drug resistant TNBC and ccRCC. An effective and safe combinational therapy would be very valuable in types of cancer where few treatment alternatives exist.

### SUMMARY

In one embodiment, provided herein are methods for diagnosis, treating, or managing a chemoresistant cancer in a patient comprising administering to said patient an effective amount of an HDAC inhibitor in combination with a DNA methyltransferase inhibitor.

HDAC inhibitors useful in the methods provided herein include, but are not limited to, trichostatin A (TSA), Vorinostat (SAHA), Valproic Acid (VPA), romidepsin and MS-275. In one embodiment, the HDAC inhibitor is romidepsin.

In one embodiment, DNA methyltransferase inhibitors useful in the methods provided herein are cytidine analogs. Cytidine analogs useful in the methods provided herein include, but are not limited to, 5-azacytidine (azacitidine), 5-azadeoxycytidine (decitabine), cytarabine, pseudoisocytidine, gemcitabine, zebularine, FCdR, Emtriva, 5,6-dihydro-5-azacytidine and procaine. In one embodiment, the cytidine analog is decitabine or azacitidine.

The chemoresistant cancers that can be treated by the methods provided herein include, but are not limited to, cancer of the skin; lymph node; breast; cervix; uterus; gastrointestinal tract; pancreas, lung; ovary; prostate; colon; rectal; mouth; brain; head and neck; throat; testes; kidney; pancreas; bone; spleen; liver; bladder; larynx; or nasal passages, and relapsed or refractory cancer. In one embodiment, the chemoresistant cancer is breast cancer, liver cancer, kidney cancer or pancreas cancer. In one specific embodiment, the cancer is triple negative breast cancer (TNBC) or clear cell renal cell carcinoma (ccRCC).

In another embodiment, provided herein is a pharmaceutical composition for diagnosis, treating, or managing a chemoresistant cancer in a patient comprising an HDAC inhibitor, a DNA methyltransferase inhibitor and a pharmaceutically acceptable carrier. In one embodiment, the HDAC inhibitor is romidepsin. In one embodiment, the DNA methyltransferase inhibitor is a cytidine analog. In one embodiment, the cytidine analog is decitabine or azacitidine.

In yet another embodiment, provided herein are single unit dosage forms, dosing regimens and kits which comprise an HDAC inhibitor and a DNA methyltransferase inhibitor. In one embodiment, the HDAC inhibitor is romidepsin. In one embodiment, a DNA methyltransferase inhibitor is a cytidine analog. In one embodiment, the cytidine analog is decitabine or azacitidine.

In one embodiment, provided herein are biomarkers for diagnosis, treating, or managing of cancers. In one embodiment, the cancer is TNBC or cRCC. In one embodiment, biomarkers useful in the methods provides herein include, but are not limited to, RhoB, p21, p15, p16, TβRIII, GATA3, sFRP1, sFRP2, sFRP4, sFRP5, DKK1 and DKK3.

In one embodiment, provided herein are biomarkers for predicting or monitoring the efficacy or clinical benefit of a therapeutic treatment in patients in need thereof, such as, in TNBC or cRCC patients treated with a combination of an HDAC inhibitor and a DNA methyltransferase inhibitor. In one embodiment, the HDAC inhibitor is romidepsin. In one embodiment, the DNA methyltransferase inhibitor is a cytidine analog. In one embodiment, the cytidine analog is decitabine or azacitidine. In one embodiment, biomarkers useful in the methods provides herein include, but are not limited to, RhoB, p21, p15, p16, TβRIII, GATA3, sFRP1, sFRP2, sFRP4, sFRP5, DKK1 and DKK3.

In one embodiment, provided herein is a method of predicting or monitoring the efficacy or clinical benefit of a therapeutic treatment, comprising measuring the level of one or more specific biomarker(s) in cells obtained from patients having a certain disease before or during the treatment. In one embodiment, the disease is cancer. In one embodiment, the cancer is chemoresistant cancer. In one embodiment, the chemoresistant cancer is TNBC or cRCC. In one embodiment, the treatment is administration of a combination of an HDAC inhibitor and a DNA methyltransferase inhibitor. In one embodiment, the HDAC inhibitor is romidepsin. In one embodiment, the DNA methyltransferase inhibitor is a cytidine analog. In one embodiment, the cytidine analog is decitabine or azacitidine.

In one embodiment, provided herein is a method of predicting or monitoring the efficacy of a combination of romidepsin and decitabine or azacitidine in TNBC or cRCC patients, comprising measuring the level of one or more specific biomarker(s) in cells obtained from patients before or during the combination therapy.

### BRIEF DESCRIPTION OF THE DRAWINGS

FIG. 1A depicts a response to romidepsin (0.01 nM to 100 nM) in ccRCC and TNBC cell lines. FIG. 1B depicts a response to decitabine (0.01 μM to 10 μM) in ccRCC and TNBC cell lines. The cell lines A498, KIJ265T, MDA-231, and BT-20 were seeded at 1×105 cells/well in triplicate per dose. Treatments were applied to cells for 72 hours prior to collection.

FIGS. 2A, 2B, 2C and 2D depict the combinatorial drug dose response to romidepsin and decitabine in ccRCC and TNBC cell lines. (A) A498, (B) KIJ265T, (C) MDA-231, and (D) BT-20 cell lines were seeded in triplicate at 1×105 cells/well for each tested drug dose. Cells were incubated with a dose of 0.1, 1, or 10 μM decitabine for 48 hours prior to being treated with a dose range of 0.5 to 7.5 nM romidepsin for an additional 24 hours. Data is presented as proliferation curves with monotherapeutic controls included.

FIGS. 3A, 3B, 3C and 3D demonstrate synergistic induction of cell death in ccRCC and TNBC cell lines treated with a combination of romidepsin and decitabine. (A) A498, (B) KIJ265T, (C) MDA-231, and (D) BT-20 cell lines treated with monotherapeutic doses of decitabine or romidepsin were analyzed versus combination treatment for drug effects leading to cell death. Cells treated with vehicle controls (DMSO) were used to set population parameters for analysis. Propidium iodide stain was applied to treated cells and analyzed via flow cytometry.

FIGS. 4A-4B depict analysis of sFRP1 expression and markers of apoptosis in ccRCC and TNBC cell lines treated with 1 μM decitabine and 5 nM romidepsin. FIG. 4A depicts immunoblots of protein lysates created from treated A498, KIJ265T, MDA-231, and BT-20 cells that were probed for PARP and caspase-3 cleavage. FIG. 4C depicts analysis of the methylation status of the sFRP1 promoter and the ability of single and combinatorial drug treatments to modulate these methylation events via methylation specific PCR using defined primers for the amplification of methylated (M) or unmethylated (U) sequences of sFRP1.

FIGS. 5A to 5G demonstrate influence of sFRP1 expression levels on cell survival in ccRCC and TNBC cell lines treated with decitabine and romidepsin. FIG. 5A is a real-time PCR of MDA-231 and KIJ265T cells infected with sFRP1 shRNA. FIGS. 5B and 5C demonstrate an increased cell survival of KIJ265T and MDA-231 cells when treated with a combinatorial dose range of decitabine and romidepsin. FIG. 5D demonstrates that attenuation of apoptosis in KIJ265T and MDA-231 sFRP1 knockdown cells is via reduction in PARP and caspase-3 cleavage versus non-target controls when treated in combination with decitabine and romidepsin. FIG. 5E demonstrates a decreased proliferation of KIJ265T and MDA-231 parent cell lines in a dose-dependent manner when treated with recombinant human sFRP1. FIG. 5F depicts the resulting loss of sFRP1 re-expression yielded an increase in cell survival of KIJ265T and MDA-231 cells when treated with a combinatorial dose of 1 μM 5A2D and 5 nM Romidepsin. FIG. 5G demonstrates decreased proliferation of KIJ265T and MDA-231 parent cell lines when observed in a dose-dependent manner when treated with recombinant human sFRP1 and verified to be through the induction of apoptosis as seen by PARP cleavage, after a single dose of 1.4 nM sFRP1.

FIGS. 6A and 6B depict authentication of the VHL mutant (Exon 2 c.407T>C) KIJ265T clear cell renal cell carcinoma cell line. FIG. 6A demonstrates STR analysis for expression of 12 renal specific markers in KIJ265T patient RCC tissue. FIG. 6B demonstrates that the KIJ265T ccRCC cell line originates from renal tissue by using IHC staining for renal cell markers including RCC-Ma, aquaporin, podocin, PAX2, and GGT.

FIGS. 7A-7D depict the methylation pattern sequencing analysis of the sFRP1 promoter region (−299 bp to −70 bp before the start site) in (A) A498, (B) KIJ265T, (C) MDA231 and (D) BT20 cell lines treated with vehicle control or 72 hours combinatorial therapy. Overall decreases in promoter methylation are observed in all cell lines after combinatorial treatment (n=10 clones).

### DETAILED DESCRIPTION

### Definitions

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. It should also be noted that use of “or” means “and/or” unless stated otherwise. Furthermore, use of the term “including” as well as other forms, such as “include,” “includes,” and “included” is not limiting.

The term “treating” as used herein, means an alleviation, in whole or in part, of symptoms associated with a disorder or disease (e.g., cancer or a tumor syndrome), or slowing, or halting of further progression or worsening of those symptoms.

The term “preventing” as used herein, means the prevention of the onset, recurrence or spread, in whole or in part, of the disease or disorder (e.g., cancer), or a symptom thereof.

The term “effective amount” in connection with the HDAC inhibitor means an amount capable of alleviating, in whole or in part, symptoms associated with a disorder, for example cancer, or slowing or halting further progression or worsening of those symptoms, or preventing or providing prophylaxis for cancer, in a subject at risk for cancer. The effective amount of the HDAC inhibitor, for example in a pharmaceutical composition, may be at a level that will exercise the desired effect; for example, about 0.005 mg/kg of a subject's body weight to about 100 mg/kg of a subject's body weight in unit dosage for both oral and parenteral administration. As will be apparent to those skilled in the art, it is to be expected that the effective amount of an HDAC inhibitor disclosed herein may vary depending on the severity of the indication being treated.

The term “pharmaceutically acceptable carrier” as used herein means a pharmaceutically acceptable material, composition or vehicle, such as a liquid or solid filler, diluent, excipient, solvent or encapsulating material, involved in carrying or transporting the subject compounds from the administration site of one organ, or portion of the body, to another organ, or portion of the body, or in an in vitro assay system. Each carrier must be “acceptable” in the sense of being compatible with the other ingredients of the formulation and not injurious to a subject to whom it is administered. Nor should an acceptable carrier alter the specific activity of the subject compounds.

The term “pharmaceutically acceptable” refers to molecular entities and compositions that are physiologically tolerable and do not typically produce an allergic or similar untoward reaction, such as gastric upset, dizziness and the like, when administered to a human.

The term “pharmaceutically acceptable salt” encompasses non-toxic acid and base addition salts of the compound to which the term refers. Acceptable non-toxic acid addition salts include those derived from organic and inorganic acids or bases know in the art, which include, for example, hydrochloric acid, hydrobromic acid, phosphoric acid, sulfuric acid, methanesulphonic acid, acetic acid, tartaric acid, lactic acid, succinic acid, citric acid, malic acid, maleic acid, sorbic acid, aconitic acid, salicylic acid, phthalic acid, embolic acid, enanthic acid, and the like.

Compounds that are acidic in nature are capable of forming salts with various pharmaceutically acceptable bases. The bases that can be used to prepare pharmaceutically acceptable base addition salts of such acidic compounds are those that form non-toxic base addition salts, i.e., salts containing pharmacologically acceptable cations such as, but not limited to, alkali metal or alkaline earth metal salts and the calcium, magnesium, sodium or potassium salts in particular. Suitable organic bases include, but are not limited to, N,N-dibenzylethylenediamine, chloroprocaine, choline, diethanolamine, ethylenediamine, meglumaine (N-methylglucamine), lysine, and procaine.

The term “prodrug” means a derivative of a compound that can hydrolyze, oxidize, or otherwise react under biological conditions (in vitro or in vivo) to provide the compound. Examples of prodrugs include, but are not limited to, derivatives of immunomodulatory compounds of the invention that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include derivatives of immunomodulatory compounds of the invention that comprise —NO, —NO₂, —ONO, or —ONO₂ moieties. Prodrugs can typically be prepared using well-known methods, such as those described in 1 Burger's Medicinal Chemistry and Drug Discovery, 172-178, 949-982 (Manfred E. Wolff ed., 5th ed. 1995), and Design of Prodrugs (H. Bundgaard ed., Elselvier, New York 1985).

The term “unit dose” when used in reference to a therapeutic composition refers to physically discrete units suitable as unitary dosage for humans, each unit containing a predetermined quantity of active material calculated to produce the desired therapeutic effect in association with the required diluent; i.e., carrier, or vehicle.

The term “unit-dosage form” refers to a physically discrete unit suitable for administration to a human and animal subject, and packaged individually as is known in the art. Each unit-dose contains a predetermined quantity of an active ingredient(s) sufficient to produce the desired therapeutic effect, in association with the required pharmaceutical carriers or excipients. A unit-dosage form may be administered in fractions or multiples thereof. Examples of a unit-dosage form include an ampoule, syringe, and individually packaged tablet and capsule.

The term “multiple-dosage form” is a plurality of identical unit-dosage forms packaged in a single container to be administered in segregated unit-dosage form. Examples of a multiple-dosage form include a vial, bottle of tablets or capsules, or bottle of pints or gallons.

The term “tumor” refers to all neoplastic cell growth and proliferation, whether malignant or benign, and all pre-cancerous and cancerous cells and tissues. As used herein, the term “neoplastic” refers to any form of dysregulated or unregulated cell growth, whether malignant or benign, resulting in abnormal tissue growth. Thus, “neoplastic cells” include malignant and benign cells having dysregulated or unregulated cell growth.

The term “cancer” includes, but is not limited to, solid tumors and blood born tumors. The term “cancer” refers to disease of skin tissues, organs, blood, and vessels, including, but not limited to, cancers of the bladder, bone or blood, brain, breast, cervix, chest, colon, endrometrium, esophagus, eye, head, kidney, liver, lymph nodes, lung, mouth, neck, ovaries, pancreas, prostate, rectum, stomach, testis, throat, and uterus.

The term “proliferative” disorder or disease refers to unwanted cell proliferation of one or more subset of cells in a multicellular organism resulting in harm (i.e., discomfort or decreased life expectancy) to the multicellular organism. For example, as used herein, proliferative disorder or disease includes neoplastic disorders and other proliferative disorders.

The term “relapsed” refers to a situation where a subject, that has had a remission of cancer after a therapy, has a return of cancer cells.

The term “refractory” or “resistant” refers to a circumstance where a subject, even after intensive treatment, has residual cancer cells in the body.

The term “chemoresistant cancer” means a type of cancer when cancer that has been responding to a therapy suddenly begins to grow because cancer cells are not responsive to the effects of chemotherapy.

The terms “active ingredient” and “active substance” refer to a compound, which is administered, alone or in combination with one or more pharmaceutically acceptable excipients, to a subject for treating, preventing, or ameliorating one or more symptoms of a condition, disorder, or disease. As used herein, “active ingredient” and “active substance” may be an optically active isomer or an isotopic variant of a compound described herein.

The terms “drug,” “therapeutic agent,” and “chemotherapeutic agent” refer to a compound, or a pharmaceutical composition thereof, which is administered to a subject for treating, preventing, or ameliorating one or more symptoms of a condition, disorder, or disease.

The terms “co-administration” and “in combination with” include the administration of two or more therapeutic agents simultaneously, concurrently or sequentially within no specific time limits unless otherwise indicated. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), essentially concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

As used herein, and unless otherwise specified, the terms “composition,” “formulation,” and “dosage form” are intended to encompass products comprising the specified ingredient(s) (in the specified amounts, if indicated), as well as any product(s) which result, directly or indirectly, from combination of the specified ingredient(s) in the specified amount(s).

The term “DNA methyltransferase inhibitor” refers to agents that inhibit the transfer of a methyl group to DNA. In one embodiment, the DNA methyltransferase inhibitors are cytidine analogs.

A cytidine analog referred to herein is intended to encompass the free base of the cytidine analog, or a salt, solvate, hydrate, cocrystal, complex, prodrug, precursor, metabolite, and/or derivative thereof. In certain embodiments, a cytidine analog referred to herein encompasses the free base of the cytidine analog, or a salt, solvate, hydrate, cocrystal or complex thereof. In certain embodiments, a cytidine analog referred to herein encompasses the free base of the cytidine analog, or a pharmaceutically acceptable salt, solvate, or hydrate thereof.

The term “hydrate” means a compound provided herein or a salt thereof, which further includes a stoichiometric or non-stoichiometric amount of water bound by non-covalent intermolecular forces.

The term “solvate” means a solvate formed from the association of one or more solvent molecules to a compound provided herein. The term “solvate” includes hydrates (e.g., hemihydrate, monohydrate, dihydrate, trihydrate, tetrahydrate, and the like).

As used herein, and unless otherwise specified, a compound described herein is intended to encompass all possible stereoisomers, unless a particular stereochemistry is specified. Where structural isomers of a compound are interconvertible via a low energy barrier, the compound may exist as a single tautomer or a mixture of tautomers. This can take the form of proton tautomerism; or so-called valence tautomerism in the compound, e.g., that contain an aromatic moiety.

In one embodiment, a compound described herein is intended to encompass isotopically enriched analogs. For example, one or more hydrogen position(s) in a compound may be enriched with deuterium and/or tritium. Other suitable isotopes that may be enriched at particular positions of a compound include, but are not limited, C-13, C-14, N-15, O-17, and/or O-18. In one embodiment, a compound described herein may be enriched at more than one position with isotopes, that are the same or different.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

Romidepsin

Romidepsin is a natural product which was isolated from Chromobacterium violaceum by Fujisawa Pharmaceuticals (Published Japanese Patent Application No. 64872, U.S. Pat. No. 4,977,138, issued Dec. 11, 1990, Ueda et al., J. Antibiot (Tokyo) 47:301-310, 1994; Nakajima et al., Exp Cell Res 241:126-133, 1998; and WO 02/20817; each of which is incorporated herein by reference. It is a bicyclic peptide consisting of four amino acid residues (D-valine, D-cysteine, dehydrobutyrine, and L-valine) and a novel acid (3-hydroxy-7-mercapto-4-heptenoic acid) containing both amide and ester bonds. In addition to the production from C. violaceum using fermentation, romidepsin can also be prepared by synthetic or semi-synthetic means. The total synthesis of romidepsin reported by Kahn et al. involves 14 steps and yields romidepsin in 18% overall yield (Kahn et al. J. Am. Chem. Soc. 118:7237-7238, 1996).

The chemical name of romidepsin is (1S,4S,7Z,10S,16E,21R)-7-ethylidene-4,21-bis(1-methylethyl)-2-oxa-12,13-dithia-5,8,20,23-tetrazabicyclo[8.7.6]tricos-16-ene-3,6,9,19,22-pentone. The empirical formula is C₂₄H₃₆N₄O₆S₂. The molecular weight is 540.71. At room temperature, romidepsin is a white powder.

It's structure is shown below (formula I):

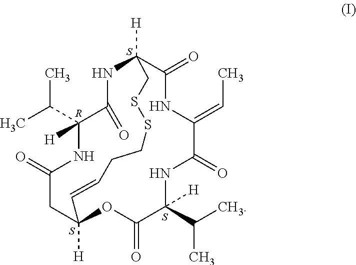

Romidepsin has been shown to have anti-microbial, immunosuppressive, and anti-tumor activities. It was tested, for example, for use in treating patients with hematological malignancies (e.g, cutaneous T-cell lymphoma (CTCL), peripheral T-cell lymphoma (PTCL), multiple myeloma, etc.) and solid tumors (e.g., prostate cancer, pancreatic cancer, etc.) and is thought to act by selectively inhibiting deacetylases (e.g., histone deacetylase, tubulin deacetylase), thus promising new targets for the development of a new class of anti-cancer therapies (Nakajima et al., Exp Cell Res 241:126-133, 1998). One mode of action of romidepsin involves the inhibition of one or more classes of histone deacetylases (HDAC). Preparations and purification of romidepsin is described, for example, in U.S. Pat. No. 4,977,138 and International PCT Application Publication WO 02/20817, each of which is incorporated herein by reference.

Exemplary forms of romidepsin include, but are not limited to, salts, esters, pro-drugs, isomers, stereoisomers (e.g., enantiomers, diastereomers), tautomers, protected forms, reduced forms, oxidized forms, derivatives, and combinations thereof, with the desired activity (e.g., deacetylase inhibitory activity, aggressive inhibition, cytotoxicity). In certain embodiments, romidepsin is a pharmaceutical grade material and meets the standards of the U.S. Pharmacopoeia, Japanese Pharmacopoeia, or European Pharmacopoeia. In certain embodiments, the romidepsin is at least 95%, at least 98%, at least 99%, at least 99.9%, or at least 99.95% pure. In certain embodiments, the romidepsin is at least 95%, at least 98%, at least 99%, at least 99.9%, or at least 99.95% monomeric. In certain embodiments, no impurities are detectable in the romidepsin materials (e.g., oxidized material, reduced material, dimerized or oligomerized material, side products, etc.). Romidepsin typically includes less than 1.0%, less than 0.5%, less than 0.2%, or less than 0.1% of total other unknowns. The purity of romidepsin may be assessed by appearance, HPLC, specific rotation, NMR spectroscopy, IR spectroscopy, UV/Visible spectroscopy, powder x-ray diffraction (XRPD) analysis, elemental analysis, LC-mass spectroscopy, or mass spectroscopy.

Romidepsin is sold under the tradename Istodax® and is approved for the treatment of cutaneous T-cell lymphoma (CTCL) in patients who have received at least one prior systemic therapy, and for the treatment of peripheral T-cell lymphoma (PTCL) in patients who have received at least one prior therapy.

DNA Demethylating Agents

In one embodiment, the methods provided herein comprise administration or co-administration of one or more DNA demethylating agents. In one embodiment, the DNA demethylating agents are cytidine analogs. In certain embodiments, the cytidine analog is 5-azacytidine (azacitidine) or 5-aza-2′-deoxycytidine (decitabine). In certain embodiments, the cytidine analog is 5-azacytidine (azacitidine). In certain embodiments, the cytidine analog is 5-aza-2′-deoxycytidine (decitabine). In certain embodiments, the cytidine analog is, for example: 1-β-D-arabinofuranosylcytosine (Cytarabine or ara-C); pseudoiso-cytidine (psi ICR); 5-fluoro-2′-deoxycytidine (FCdR); 2′-deoxy-2′,2′-difluorocytidine (Gemcitabine); 5-aza-2′-deoxy-2′,2′-difluorocytidine; 5-aza-2′-deoxy-2′-fluorocytidine; 1-β-D-ribofuranosyl-2(1H)-pyrimidinone (Zebularine); 2′,3′-dideoxy-5-fluoro-3′-thiacytidine (Emtriva); 2′-cyclocytidine (Ancitabine); 1-β-D-arabinofuranosyl-5-azacytosine (Fazarabine or ara-AC); 6-azacytidine (6-aza-CR); 5,6-dihydro-5-azacytidine (dH-aza-CR); N⁴-pentyloxy-carbonyl-5′-deoxy-5-fluorocytidine (Capecitabine); N⁴-octadecyl-cytarabine; or elaidic acid cytarabine. In certain embodiments, the cytidine analogs provided herein include any compound which is structurally related to cytidine or deoxycytidine and functionally mimics and/or antagonizes the action of cytidine or deoxycytidine.

In certain embodiments, exemplary cytidine analogs have the structures provided below:

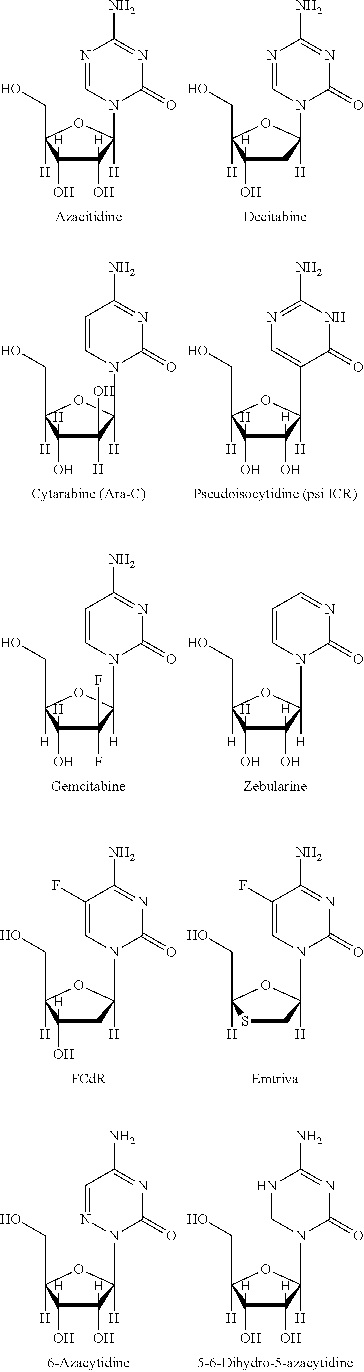

Certain embodiments herein provide salts, co-crystals, solvates (e.g., hydrates), complexes, prodrugs, precursors, metabolites, and/or other derivatives of the cytidine analogs provided herein. For example, particular embodiments provide salts, cocrystals, solvates (e.g., hydrates), complexes, precursors, metabolites, and/or other derivatives of 5-azacytidine. Certain embodiments herein provide salts, cocrystals, and/or solvates (e.g., hydrates) of the cytidine analogs provided herein. Certain embodiments herein provide salts and/or solvates (e.g., hydrates) of the cytidine analogs provided herein. Certain embodiments provide cytidine analogs that are not salts, cocrystals, solvates (e.g., hydrates), or complexes of the cytidine analogs provided herein. For example, particular embodiments provide 5-azacytidine in a non-ionized, non-solvated (e.g., anhydrous), non-complexed form. Certain embodiments herein provide a mixture of two or more cytidine analogs provided herein.

Cytidine analogs provided herein may be prepared using synthetic methods and procedures referenced herein or otherwise available in the literature. For example, particular methods for synthesizing 5-azacytidine and decitabine are disclosed, e.g., in U.S. Pat. No. 7,038,038 and references discussed therein, each of which is incorporated herein by reference. Other cytidine analogs provided herein may be prepared, e.g., using procedures known in the art, or may be purchased from a commercial source. In one embodiment, the cytidine analogs provided herein may be prepared in a particular solid form (e.g., amorphous or crystalline form). See, e.g., U.S. Pat. No. 6,887,855, issued May 8, 2005 and U.S. Pat. No. 6,943,249, issued Sep. 13, 2005, both of which are incorporated herein by reference in their entireties.

In one embodiment, the compound used in the methods provided herein is a free base, or a pharmaceutically acceptable salt or solvate thereof. In one embodiment, the free base or the pharmaceutically acceptable salt or solvate is a solid. In another embodiment, the free base or the pharmaceutically acceptable salt or solvate is a solid in an amorphous form. In yet another embodiment, the free base or the pharmaceutically acceptable salt or solvate is a solid in a crystalline form. For example, particular embodiments provide 5-azacytidine and decitabine in solid forms, which can be prepared, for example, according to the methods described in U.S. Pat. Nos. 6,943,249, 6,887,855, 7,078,518, 7,772,199 and U.S. Patent Application Publication Nos. 2005/027675, each of which is incorporated by reference herein in their entireties. In other embodiments, 5-azacytidine and decitabine in solid forms can be prepared using other methods known in the art.

In one embodiment, the compound used in the methods provided herein is a pharmaceutically acceptable salt of the cytidine analog, which includes, but is not limited to, acetate, adipate, alginate, aspartate, benzoate, benzenesulfonate (besylate), bisulfate, butyrate, citrate, camphorate, camphorsulfonate, cyclopentanepropionate, digluconate, dodecylsulfate, 1,2-ethanedisulfonate (edisylate), ethanesulfonate (esylate), formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate (mesylate), 2-naphthalenesulfonate (napsylate), nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate, or undecanoate salts.

Cytidine analogs may be synthesized by methods known in the art. In one embodiment, methods of synthesis include methods as disclosed in U.S. Pat. No. 7,038,038; U.S. Pat. No. 6,887,855; U.S. Pat. No. 7,078,518; U.S. Pat. No. 6,943,249; and U.S. Pat. No. 7,192,781, all incorporated by reference herein in their entireties.

Azacitidine is 4-amino-1-β-D-ribofuranozyl-s-triazin-2(1H)-one, also known as VIDAZA®. Its empirical formula is C₈H₁₂N₄O₅, the molecular weight is 244. Azacitidine is a white to off-white solid that is insoluble in acetone, ethanol and methyl ketone; slightly soluble in ethanol/water (50/50), propylene glycol and polyethylene glycol; sparingly soluble in water, water-saturated octanol, 5% dextrose in water, N-methyl-2-pyrrolidone, normal saline and 5% Tween 80 in water, and soluble in dimethylsulfoxide (DMSO).

VIDAZA® is approved for treatment in patients with higher-risk MDS. It is supplied in a sterile form for reconstitution as a suspension for subcutaneous injection or reconstitution as a solution with further dilution for intravenous infusion. Vials of VIDAZA® contain 100 mg of azacitidine and 100 mg of mannitol as a sterile lyophilized powder.

Decitabine is 4-amino-1-(2-deoxy-β-D-erythro-pentofuranosyl)-1,3,5-triazin-2(1H)one, also known as DACOGEN®™. Its empirical formula is C₈H₁₂N₄O₄, the molecular weight is 228.21. Decitabine is a fine, white to almost white powder that is slightly soluble in ethanol/water (50/50), methanol/water (50/50) and methanol; sparingly soluble in water, and soluble in dimethylsulfoxide (DMSO).

DACOGEN™ is approved for treatment in patients with myelodisplastic syndromes. It is supplied in a clear colorless glass vial as white sterile lyophilized powder for injection. Each 20 mL, as a single dose, glass vial contains 50 mg decitabine, 68 mg monobasic potassium phosphate (potassium dihydrogen phosphate) and 11.6 mg sodium hydrochloride.

Methods of Use

In one embodiment, provided is a method for treating, preventing, or managing TNBC or ccRCC in a patient comprising administering to said patient an effective amount of HDAC inhibitor in combination with a DNA demethylating agent or a pharmaceutically acceptable salt, solvate, hydrate, stereoisomer, clathrate, or prodrug thereof.

HDAC inhibitors for use in the methods provided herein include, but are not limited to, trichostatin A (TSA), Vorinostat (SAHA), Valproic Acid (VPA), romidepsin and MS-275. In one embodiment, the HDAC inhibitor is romidepsin.

The DNA demethylating agents useful in the methods provided herein are cytidine analogs. In one embodiment, cytidine analogs include, but are not limited to, 5-azacytidine (azacitidine), 5-azadeoxycytidine (decitabine), cytarabine, pseudoisocytidine, gemcitabine, zebularine, FCdR, Emtriva, 5,6-dihydro-5-azacytidine and procaine. In one embodiment, the cytidine analog is decitabine or azacitidine.

The chemoresistant cancers that can be treated by the methods provided herein include, but are not limited to, cancer of the skin; lymph node; breast; cervix; uterus; gastrointestinal tract; pancreas, lung; ovary; prostate; colon; rectal; mouth; brain; head and neck; throat; testes; kidney; pancreas; bone; spleen; liver; bladder; larynx; or nasal passages, and relapsed or refractory cancer. In one embodiment, the chemoresistant cancer is breast cancer, liver cancer, kidney cancer or pancreas cancer. In one specific embodiment, the cancer is triple negative breast cancer (TNBC) or clear cell renal cell carcinoma (ccRCC).

Administration of romidepsin and decitabine or azacitidine can occur simultaneously or sequentially by the same or different routes of administration. The suitability of a particular route of administration employed for a particular active agent will depend on the active agent itself (e.g., whether it can be administered orally without decomposing prior to entering the blood stream) and the disease being treated.

Suitable routes of administration include, but are not limited to, oral, mucosal (e.g., nasal, sublingual, vaginal, buccal, or rectal), parenteral (e.g., subcutaneous, intravenous, bolus injection, intramuscular, or intraarterial), topical (e.g., eye drops or other ophthalmic preparations), transdermal or transcutaneous administration to a patient.

In one embodiment, an effective amount of romidepsin and decitabine or azacitidine to be used is a therapeutically effective amount. In one embodiment, the amounts of romidepsin and decitabine or azacitidine to be used in the methods provided herein include an amount sufficient to cause improvement in at least a subset of patients with respect to symptoms, overall course of disease, or other parameters known in the art. Precise amounts for therapeutically effective amounts of romidepsin or azacitidine in the pharmaceutical compositions will vary depending on the age, weight, disease, and condition of the patient.

In one embodiment, romidepsin is administered intravenously. In one embodiment, romidepsin is administered intravenously over a 1-6 hour period. In one embodiment, romidepsin is administered intravenously over a 3-4 hour period. In one embodiment, romidepsin is administered intravenously over a 5-6 hour period. In one embodiment, romidepsin is administered intravenously over a 4 hour period.

In one embodiment, romidepsin is administered in a dose ranging from 0.5 mg/m² to 28 mg/m². In one embodiment, romidepsin is administered in a dose ranging from 0.5 mg/m² to 5 mg/m². In one embodiment, romidepsin is administered in a dose ranging from 1 mg/m² to 25 mg/m². In one embodiment, romidepsin is administered in a dose ranging from 1 m g/m² to 20 mg/m². In one embodiment, romidepsin is administered in a dose ranging from 1 mg/m² to 15 mg/m². In one embodiment, romidepsin is administered in a dose ranging from 2 mg/m² to 15 mg/m². In one embodiment, romidepsin is administered in a dose ranging from 2 mg/m² to 12 mg/m². In one embodiment, romidepsin is administered in a dose ranging from 4 mg/m² to 12 mg/m². In one embodiment, romidepsin is administered in a dose ranging from 6 mg/m² to 12 mg/m². In one embodiment, romidepsin is administered in a dose ranging from 8 mg/m² to 12 mg/m². In one embodiment, romidepsin is administered in a dose ranging from 8 mg/m² to 10 mg/m². In one embodiment, romidepsin is administered in a dose of about 8 mg/m². In one embodiment, romidepsin is administered in a dose of about 9 mg/m². In one embodiment, romidepsin is administered in a dose of about 10 mg/m². In one embodiment, romidepsin is administered in a dose of about 11 mg/m². In one embodiment, romidepsin is administered in a dose of about 12 mg/m². In one embodiment, romidepsin is administered in a dose of about 13 mg/m². In one embodiment, romidepsin is administered in a dose of about 14 mg/m². In one embodiment, romidepsin is administered in a dose of about 15 mg/m².

In one embodiment, romidepsin is administered in a dose of 14 mg/m² over a 4 hour iv infusion on days 1, 8 and 15 of the 28 day cycle. In one embodiment, the cycle is repeated every 28 days.

In one embodiment, increasing doses of romidepsin are administered over the course of a cycle. In one embodiment, the dose of about 8 mg/m² followed by a dose of about 10 mg/m², followed by a dose of about 12 mg/m² is administered over a cycle.

In one embodiment, romidepsin is administered orally. In one embodiment, romidepsin is administered in a dose ranging from 10 mg/m² to 300 mg/m². In one embodiment, romidepsin is administered in a dose ranging from 15 mg/m² to 250 mg/m². In one embodiment, romidepsin is administered in a dose ranging from 20 mg/m² to 200 mg/m². In one embodiment, romidepsin is administered in a dose ranging from 25 m g/m² to 150 mg/m². In one embodiment, romidepsin is administered in a dose ranging from 25 mg/m² to 100 mg/m². In one embodiment, romidepsin is administered in a dose ranging from 25 mg/m² to 75 mg/m².

In one embodiment, romidepsin is administered orally on a daily basis. In one embodiment, romidepsin is administered orally every other day. In one embodiment, romidepsin is administered orally every third, fourth, fifth, or sixth day. In one embodiment, romidepsin is administered orally every week. In one embodiment, romidepsin is administered orally every other week.

In one embodiment, decitabine or azacitidine is administered by, e.g., intravenous (IV), subcutaneous (SC) or oral routes. Certain embodiments herein provide co-administration of decitabine or azacitidine with one or more additional active agents to provide a synergistic therapeutic effect in subjects in need thereof. The co-administered agent(s) may be a cancer therapeutic agent, as described herein. In certain embodiments, the co-administered agent(s) may be dosed, e.g., orally or by injection (e.g., IV or SC).

In certain embodiments, treatment cycles comprise multiple doses administered to a subject in need thereof over multiple days (e.g., 1, 2, 3, 4, 5, 6, 7, 8, 9, 10, 11, 12, 13, 14, or greater than 14 days), optionally followed by treatment dosing holidays (e.g., 1, 2, 3, 4, 5, 6, 7, 8, 9, 10, 11, 12, 13, 14, 15, 16, 17, 18, 19, 20, 21, 22, 23, 24, 25, 26, 27, 28, or greater than 28 days). Suitable dosage amounts for the methods provided herein include, e.g., therapeutically effective amounts and prophylactically effective amounts. For example, in certain embodiments, the amount of decitabine or azacitidine administered in the methods provided herein may range, e.g., between about 10 mg/m²/day and about 2,000 mg/m²/day, between about 100 mg/m²/day and about 1,000 mg/m²/day, between about 100 mg/m²/day and about 500 mg/m²/day, between about 50 mg/m²/day and about 500 mg/m²/day, between about 50 mg/m²/day and about 200 m g/m²/day, between about 50 mg/m²/day and about 100 mg/m²/day, between about 50 mg/m²/day and about 75 mg/m²/day, or between about 120 mg/m²/day and about 250 mg/m²/day. In certain embodiments, particular dosages are, e.g., about 50 mg/m²/day, about 60 mg/m²/day, about 75 mg/m²/day, about 80 mg/m²/day, about 100 mg/m²/day, about 120 mg/m²/day, about 140 mg/m²/day, about 150 mg/m²/day, about 180 mg/m²/day, about 200 mg/m²/day, about 220 mg/m²/day, about 240 mg/m²/day, about 250 mg/m²/day, about 260 mg/m²/day, about 280 mg/m²/day, about 300 mg/m²/day, about 320 mg/m²/day, about 350 mg/m²/day, about 380 mg/m²/day, about 400 mg/m²/day, about 450 mg/m²/day, or about 500 mg/m²/day. In certain embodiments, particular dosages are, e.g., up to about 100 mg/m²/day, up to about 120 mg/m²/day, up to about 140 mg/m²/day, up to about 150 mg/m²/day, up to about 180 mg/m²/day, up to about 200 mg/m²/day, up to about 220 mg/m²/day, up to about 240 mg/m²/day, up to about 250 mg/m²/day, up to about 260 mg/m²/day, up to about 280 mg/m²/day, up to about 300 mg/m²/day, up to about 320 mg/m²/day, up to about 350 mg/m²/day, up to about 380 mg/m²/day, up to about 400 mg/m²/day, up to about 450 mg/m²/day, up to about 500 mg/m²/day, up to about 750 mg/m²/day, or up to about 1000 mg/m²/day.

In one embodiment, the amount of decitabine or azacitidine administered in the methods provided herein may range, e.g., between about 5 mg/day and about 2,000 mg/day, between about 10 mg/day and about 2,000 mg/day, between about 20 mg/day and about 2,000 mg/day, between about 50 mg/day and about 1,000 mg/day, between about 100 mg/day and about 1,000 mg/day, between about 100 mg/day and about 500 mg/day, between about 150 mg/day and about 500 mg/day, or between about 150 mg/day and about 250 mg/day. In certain embodiments, particular dosages are, e.g., about 10 mg/day, about 20 mg/day, about 50 mg/day, about 75 mg/day, about 100 mg/day, about 120 mg/day, about 150 mg/day, about 200 mg/day, about 250 mg/day, about 300 mg/day, about 350 mg/day, about 400 mg/day, about 450 mg/day, about 500 mg/day, about 600 mg/day, about 700 mg/day, about 800 mg/day, about 900 mg/day, about 1,000 mg/day, about 1,200 mg/day, or about 1,500 mg/day. In certain embodiments, particular dosages are, e.g., up to about 10 mg/day, up to about 20 mg/day, up to about 50 mg/day, up to about 75 mg/day, up to about 100 mg/day, up to about 120 mg/day, up to about 150 mg/day, up to about 200 mg/day, up to about 250 mg/day, up to about 300 mg/day, up to about 350 mg/day, up to about 400 mg/day, up to about 450 mg/day, up to about 500 mg/day, up to about 600 mg/day, up to about 700 mg/day, up to about 800 mg/day, up to about 900 mg/day, up to about 1,000 mg/day, up to about 1,200 mg/day, or up to about 1,500 mg/day.

In one embodiment, the amount of decitabine or azacitidine in the pharmaceutical composition or dosage form provided herein may range, e.g., between about 5 mg and about 2,000 mg, between about 10 mg and about 2,000 mg, between about 20 mg and about 2,000 mg, between about 50 mg and about 1,000 mg, between about 50 mg and about 500 mg, between about 50 mg and about 250 mg, between about 100 mg and about 500 mg, between about 150 mg and about 500 mg, or between about 150 mg and about 250 mg. In certain embodiments, particular amounts are, e.g., about 10 mg, about 20 mg, about 50 mg, about 75 mg, about 100 mg, about 120 mg, about 150 mg, about 200 mg, about 250 mg, about 300 mg, about 350 mg, about 400 mg, about 450 mg, about 500 mg, about 600 mg, about 700 mg, about 800 mg, about 900 mg, about 1,000 mg, about 1,200 mg, or about 1,500 mg. In certain embodiments, particular amounts are, e.g., up to about 10 mg, up to about 20 mg, up to about 50 mg, up to about 75 mg, up to about 100 mg, up to about 120 mg, up to about 150 mg, up to about 200 mg, up to about 250 mg, up to about 300 mg, up to about 350 mg, up to about 400 mg, up to about 450 mg, up to about 500 mg, up to about 600 mg, up to about 700 mg, up to about 800 mg, up to about 900 mg, up to about 1,000 mg, up to about 1,200 mg, or up to about 1,500 mg.

In one embodiment, depending on the disease to be treated and the subject's condition, decitabine or azacitidine may be administered by oral, parenteral (e.g., intramuscular, intraperitoneal, intravenous, CIV, intracistemal injection or infusion, subcutaneous injection, or implant), inhalation, nasal, vaginal, rectal, sublingual, or topical (e.g., transdermal or local) routes of administration. Decitabine or azacitidine may be formulated, alone or together with one or more active agent(s), in suitable dosage unit with pharmaceutically acceptable excipients, carriers, adjuvants and vehicles, appropriate for each route of administration. In one embodiment, decitabine or azacitidine is administered orally. In another embodiment, decitabine or azacitidine is administered parenterally. In yet another embodiment, decitabine or azacitidine is administered intravenously.

In one embodiment, 5 decitabine or azacitidine can be delivered as a single dose such as, e.g., a single bolus injection, or oral tablets or pills; or over time such as, e.g., continuous infusion over time or divided bolus doses over time. In one embodiment, decitabine or azacitidine can be administered repetitively if necessary, for example, until the patient experiences stable disease or regression, or until the patient experiences disease progression or unacceptable toxicity. For example, stable disease for solid tumors generally means that the perpendicular diameter of measurable lesions has not increased by 25% or more from the last measurement. See, e.g., Response Evaluation Criteria in Solid Tumors (RECIST) Guidelines, Journal of the National Cancer Institute 92(3): 205-216 (2000). Stable disease or lack thereof is determined by methods known in the art such as evaluation of patient's symptoms, physical examination, visualization of the tumor that has been imaged using X-ray, CAT, PET, or MRI scan and other commonly accepted evaluation modalities.

In one embodiment, decitabine or azacitidine can be administered once daily or divided into multiple daily doses such as twice daily, three times daily, and four times daily. In one embodiment, the administration can be continuous (i.e., daily for consecutive days or every day), intermittent, e.g., in cycles (i.e., including days, weeks, or months of rest when no drug is administered). In one embodiment, decitabine or azacitidine is administered daily, for example, once or more than once each day for a period of time. In one embodiment, decitabine or azacitidine is administered daily for an uninterrupted period of at least 7 days, in some embodiments, up to 52 weeks. In one embodiment, decitabine or azacitidine is administered intermittently, i.e., stopping and starting at either regular or irregular intervals. In one embodiment, decitabine or azacitidine is administered for one to six days per week. In one embodiment, decitabine or azacitidine is administered in cycles (e.g., daily administration for two to eight consecutive weeks, then a rest period with no administration for up to one week; or e.g., daily administration for one week, then a rest period with no administration for up to three weeks). In one embodiment, decitabine or azacitidine is administered on alternate days. In one embodiment, decitabine or azacitidine is administered in cycles (e.g., administered daily or continuously for a certain period interrupted with a rest period).

In one embodiment, the frequency of administration ranges from about daily to about monthly. In certain embodiments, decitabine or azacitidine is administered once a day, twice a day, three times a day, four times a day, once every other day, twice a week, once every week, once every two weeks, once every three weeks, or once every four weeks. In one embodiment, decitabine or azacitidine is administered once a day. In another embodiment, decitabine or azacitidine is administered twice a day. In yet another embodiment, decitabine or azacitidine is administered three times a day. In still another embodiment, decitabine or azacitidine is administered four times a day.

In one embodiment, decitabine or azacitidine is administered once per day from one day to six months, from one week to three months, from one week to four weeks, from one week to three weeks, or from one week to two weeks. In certain embodiments, decitabine or azacitidine is administered once per day for one week, two weeks, three weeks, or four weeks. In one embodiment, decitabine or azacitidine is administered once per day for one week. In another embodiment, decitabine or azacitidine is administered once per day for two weeks. In yet another embodiment, decitabine or azacitidine is administered once per day for three weeks. In still another embodiment, decitabine or azacitidine is administered once per day for four weeks.

In one embodiment, decitabine or azacitidine is administered once per day for about 1 week, about 2 weeks, about 3 weeks, about 4 weeks, about 6 weeks, about 9 weeks, about 12 weeks, about 15 weeks, about 18 weeks, about 21 weeks, or about 26 weeks. In certain embodiments, decitabine or azacitidine is administered intermittently. In certain embodiments, decitabine or azacitidine is administered intermittently in the amount of between about 50 mg/m²/day and about 2,000 mg/m²/day. In certain embodiments, decitabine or azacitidine is administered continuously. In certain embodiments, decitabine or azacitidine is administered continuously in the amount of between about 50 mg/m²/day and about 1,000 mg/m²/day.

In certain embodiments, decitabine or azacitidine is administered to a patient in cycles (e.g., daily administration for one week, then a rest period with no administration for up to three weeks). Cycling therapy involves the administration of an active agent for a period of time, followed by a rest for a period of time, and repeating this sequential administration. Cycling therapy can reduce the development of resistance, avoid or reduce the side effects, and/or improves the efficacy of the treatment.

In one embodiment, decitabine or azacitidine is administered to a patient in cycles. In one embodiment, a method provided herein comprises administering decitabine or azacitidine in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or greater than 40 cycles. In one embodiment, the median number of cycles administered in a group of patients is about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or greater than about 30 cycles.

In one embodiment, decitabine or azacitidine is administered to a patient at a dose provided herein over a cycle of 28 days which consists of a 7-day treatment period and a 21-day resting period. In one embodiment, decitabine or azacitidine is administered to a patient at a dose provided herein each day from day 1 to day 7, followed with a resting period from day 8 to day 28 with no administration of decitabine or azacitidine. In one embodiment, decitabine or azacitidine is administered to a patient in cycles, each cycle consisting of a 7-day treatment period followed with a 21-day resting period. In particular embodiments, decitabine or azacitidine is administered to a patient at a dose of about 50, about 60, about 70, about 75, about 80, about 90, or about 100 mg/m²/day, for 7 days, followed with a resting period of 21 days. In one embodiment, decitabine or azacitidine is administered intravenously. In one embodiment, decitabine or azacitidine is administered subcutaneously.

In other embodiments, decitabine or azacitidine is administered orally in cycles.

Accordingly, in one embodiment, decitabine or azacitidine is administered daily in single or divided doses for about one week, about two weeks, about three weeks, about four weeks, about five weeks, about six weeks, about eight weeks, about ten weeks, about fifteen weeks, or about twenty weeks, followed by a rest period of about 1 day to about ten weeks. In one embodiment, the methods provided herein contemplate cycling treatments of about one week, about two weeks, about three weeks, about four weeks, about five weeks, about six weeks, about eight weeks, about ten weeks, about fifteen weeks, or about twenty weeks. In some embodiments, decitabine or azacitidine is administered daily in single or divided doses for about one week, about two weeks, about three weeks, about four weeks, about five weeks, or about six weeks with a rest period of about 1, 3, 5, 7, 9, 12, 14, 16, 18, 20, 22, 24, 26, 28, 29, or 30 days. In some embodiments, the rest period is 1 day. In some embodiments, the rest period is 3 days. In some embodiments, the rest period is 7 days. In some embodiments, the rest period is 14 days. In some embodiments, the rest period is 28 days. The frequency, number and length of dosing cycles can be increased or decreased.

In one embodiment, the methods provided herein comprise: i) administering to the subject a first daily dose of decitabine or azacitidine; ii) optionally resting for a period of at least one day where decitabine or azacitidine is not administered to the subject; iii) administering a second dose of decitabine or azacitidine to the subject; and iv) repeating steps ii) to iii) a plurality of times. In certain embodiments, the first daily dose is between about 50 mg/m²/day and about 2,000 mg/m²/day. In certain embodiments, the second daily dose is between about 50 mg/m²/day and about 2,000 mg/m²/day. In certain embodiments, the first daily dose is higher than the second daily dose. In certain embodiments, the second daily dose is higher than the first daily dose. In one embodiment, the rest period is 2 days, 3 days, 5 days, 7 days, 10 days, 12 days, 13 days, 14 days, 15 days, 17 days, 21 days, or 28 days. In one embodiment, the rest period is at least 2 days and steps ii) through iii) are repeated at least three times. In one embodiment, the rest period is at least 2 days and steps ii) through iii) are repeated at least five times. In one embodiment, the rest period is at least 3 days and steps ii) through iii) are repeated at least three times. In one embodiment, the rest period is at least 3 days and steps ii) through iii) are repeated at least five times. In one embodiment, the rest period is at least 7 days and steps ii) through iii) are repeated at least three times. In one embodiment, the rest period is at least 7 days and steps ii) through iii) are repeated at least five times. In one embodiment, the rest period is at least 14 days and steps ii) through iii) are repeated at least three times. In one embodiment, the rest period is at least 14 days and steps ii) through iii) are repeated at least five times. In one embodiment, the rest period is at least 21 days and steps ii) through iii) are repeated at least three times. In one embodiment, the rest period is at least 21 days and steps ii) through iii) are repeated at least five times. In one embodiment, the rest period is at least 28 days and steps ii) through iii) are repeated at least three times. In one embodiment, the rest period is at least 28 days and steps ii) through iii) are repeated at least five times. In one embodiment, the methods provided herein comprise: i) administering to the subject a first daily dose of decitabine or azacitidine for 1, 2, 3, 4, 5, 6, 7, 8, 9, 10, 11, 12, 13, or 14 days; ii) resting for a period of 1, 2, 3, 4, 5, 6, 7, 8, 9, 10, 11, 12, 13, 14, 15, 16, 17, 18, 19, 20, 21, 22, 23, 24, 25, 26, 27, or 28 days; iii) administering to the subject a second daily dose of decitabine or azacitidine for 1, 2, 3, 4, 5, 6, 7, 8, 9, 10, 11, 12, 13, or 14 days; and iv) repeating steps ii) to iii) a plurality of times. In one embodiment, the methods provided herein comprise: i) administering to the subject a daily dose of decitabine or azacitidine for 1, 2, 3, 4, 5, 6, 7, 8, 9, 10, 11, 12, 13, or 14 days; ii) resting for a period of 1, 2, 3, 4, 5, 6, 7, 8, 9, 10, 11, 12, 13, 14, 15, 16, 17, 18, 19, 20, 21, 22, 23, 24, 25, 26, 27, or 28 days; and iii) repeating steps i) to ii) a plurality of times. In one embodiment, the methods provided herein comprise: i) administering to the subject a daily dose of decitabine or azacitidine for 7 days; ii) resting for a period of 21 days; and iii) repeating steps i) to ii) a plurality of times. In one embodiment, the daily dose is between about 50 mg/m²/day and about 2,000 mg/m²/day. In one embodiment, the daily dose is between about 50 mg/m²/day and about 1,000 mg/m²/day. In one embodiment, the daily dose is between about 50 mg/m²/day and about 500 mg/m²/day. In one embodiment, the daily dose is between about 50 mg/m²/day and about 200 mg/m²/day. In one embodiment, the daily dose is between about 50 mg/m²/day and about 100 mg/m²/day.

In certain embodiments, decitabine or azacitidine is administered continuously for between about 1 and about 52 weeks. In certain embodiments, decitabine or azacitidine is administered continuously for about 1, 2, 3, 4, 5, 6, 7, 8, 9, 10, 11, or 12 months. In certain embodiments, decitabine or azacitidine is administered continuously for about 14, about 28, about 42, about 84, or about 112 days. It is understood that the duration of the treatment may vary with the age, weight, and condition of the subject being treated, and may be determined empirically using known testing protocols or according to the professional judgment of the person providing or supervising the treatment. The skilled clinician will be able to readily determine, without undue experimentation, an effective drug dose and treatment duration, for treating an individual subject having a particular type of cancer.

In one embodiment, pharmaceutical compositions may contain sufficient quantities of decitabine or azacitidine to provide a daily dosage of about 10 to 150 mg/m² (based on patient body surface area) or about 0.1 to 4 mg/kg (based on patient body weight) as single or divided (2-3) daily doses. In one embodiment, dosage is provided via a seven-day administration of 75 mg/m² subcutaneously, once every twenty-eight days, for as long as clinically necessary. In one embodiment, dosage is provided via a seven-day administration of 100 mg/m² subcutaneously, once every twenty-eight days, for as long as clinically necessary. In one embodiment, up to 4, up to 5, up to 6, up to 7, up to 8, up to 9 or more 28-day cycles are administered. Other methods for providing an effective amount of decitabine or azacitidine are disclosed in, for example, “Colon-Targeted Oral Formulations of Cytidine Analogs”, U.S. Ser. No. 11/849,958, and “Oral Formulations of Cytidine Analogs and Methods of Use Thereof”, U.S. Ser. No. 12/466,213, both of which are incorporated by reference herein in their entireties.

In particular embodiments, the number of cycles administered is, e.g., at least 4, at least 5, at least 6, at least 7, at least 8, at least 9, at least 10, at least 11, at least 12, at least 13, at least 14, at least 15, at least 16, at least 17, at least 18, at least 19, at least 20, at least 22, at least 24, at least 26, at least 28, at least 30, at least 32, at least 34, at least 36, at least 38, at least 40, at least 42, at least 44, at least 46, at least 48, or at least 50 cycles of decitabine or azacitidine treatment. In particular embodiments, the treatment is administered, e.g., 1, 2, 3, 4, 5, 6, 7, 8, 9, 10, 11, 12, 13, or 14 days out of a 28-day period. In particular embodiments, the decitabine or azacitidine dose is, e.g., at least 10 mg/day, at least 20 mg/day, at least 30 mg/day, at least 40 mg/day, at least 50 mg/day, at least 55 mg/day, at least 60 mg/day, at least 65 mg/day, at least 70 mg/day, at least 75 mg/day, at least 80 mg/day, at least 85 mg/day, at least 90 mg/day, at least 95 mg/day, or at least 100 mg/day.

In particular embodiments, the dosing is performed, e.g., subcutaneously or intravenously. In particular embodiments, the contemplated specific decitabine or azacitidine dose is, e.g., at least 10 mg/m²/day, at least 15 mg/m²/day, at least 20 mg/m²/day, at least 25 mg/m²/day, at least 50 mg/m²/day, at least 60 mg/m²/day, at least 70 mg/m²/day, at least 75 mg/m²/day, at least 80 mg/m2/day, at least 90 mg/m²/day, or at least 100 mg/m²/day. One particular embodiment herein provides administering the treatment for 3 days out of each 28-day period. One particular embodiment herein provides administering the treatment for 7 days out of each 28-day period. One particular embodiment herein provides a dosing regimen of 15 mg/m² intravenously, every 8 hours for 73 days. One particular embodiment herein provides a dosing regimen of 75 mg/m² subcutaneously or intravenously, daily for 7 days. One particular embodiment herein provides a dosing regimen of 100 mg/m² subcutaneously or intravenously, daily for 7 days.

In one embodiment, romidepsin and decitabine or azacitidine are administered intravenously. In one embodiment, the combination is administered intravenously over a 1-6 hour period. In one embodiment, the combination is administered intravenously over a 3-4 hour period. In one embodiment, the combination is administered intravenously over a 5-6 hour period. In one embodiment, the combination is administered intravenously over a 4 hour period.

In one embodiment, the combination with increasing doses of romidepsin is administered over the course of a cycle. In one embodiment, the dose of about 8 mg/m² followed by a dose of about 10 mg/m², followed by a dose of about 12 mg/m² of romidepsin is administered over a cycle.

In one embodiment, romidepsin is administered intravenously and decitabine or azacitidine is administered subcutaneously. In one embodiment, romidepsin is administered intravenously and decitabine or azacitidine is administered orally. In one embodiment, romidepsin and decitabine or azacitidine are administered orally.

In one embodiment, decitabine or azacitidine is administered daily based on 3 to 14 days administration every 28-day cycle in a single or divided doses in a four to forty week period with a rest period of about a week or two weeks. In one embodiment, decitabine or azacitidine is administered daily based on 7 to 14 days administration every 28-day cycle in a single or divided doses in a four to forty week period with a rest period of about a week or two weeks.

In one embodiment, decitabine or azacitidine is administered daily and continuously for four to forty weeks at a dose of from about 10 to about 150 mg/m² followed by a break of one or two weeks. In a particular embodiment, decitabine or azacitidine is administered in an amount of from about 0.1 to about 4.0 mg/day for four to forty weeks, with one week or two weeks of rest in a four or six week cycle.

In one embodiment, decitabine or azacitidine is administered intravenously to patients with TNBC or ccRCC in an amount of from about 0.1 to about 4.0 mg per day for about 3 to about 14 days followed by about 14 to about 25 days of rest in a 28 day cycle combined with romidepsin administered intravenously in a dose of about 0.5 mg/m² to about 28 mg/m² administered on days 1, 8 and 15 of the 28 day cycle.

In one embodiment, decitabine or azacitidine is administered intravenously to patients with TNBC or ccRCC in an amount of from about 0.10 to about 4.0 mg per day for about 3 to about 14 days followed by about 14 to about 25 day of rest in a 28 day cycle combined with romidepsin administered orally in a dose of about 10 mg/m² to about 300 mg/m² administered on days 1, 8 and 15 of the 28 day cycle.

In one embodiment, decitabine or azacitidine is administered subcutaneously to patients with TNBC or ccRCC in an amount of from about 0.10 to about 4.0 mg per day for about 3 to about 14 days followed by about 14 to about 25 day of rest in a 28 day cycle combined with romidepsin administered intravenously in a dose of about 10 mg/m² to about 300 mg/m² administered on days 1, 8 and 15 of the 28 day cycle.

In one embodiment, decitabine or azacitidine is administered subcutaneously to patients with TNBC or ccRCC in an amount of from about 0.10 to about 4.0 mg per day for about 3 to about 14 days followed by about 14 to about 25 day of rest in a 28 day cycle combined with romidepsin administered orally in a dose of about 10 mg/m² to about 300 mg/m² administered on days 1, 8 and 15 of the 28 day cycle.

In one embodiment, decitabine or azacitidine is administered orally to patients with TNBC or ccRCC in an amount of from about 0.10 to about 4.0 mg per day for about 3 to about 14 days followed by about 14 to about 25 day of rest in a 28 day cycle combined with romidepsin administered orally in a dose of about 10 mg/m² to about 300 mg/m² administered on days 1, 8 and 15 of the 28 day cycle.

In one embodiment, decitabine or azacitidine and romidepsin are administered intravenously, with administration of romidepsin occurring 30 to 60 minutes prior to decitabine or azacitidine during a cycle of four to forty weeks. In another embodiment, decitabine or azacitidine is administered subcutaneously and romidepsin is administered by intravenous infusion. In another embodiment, decitabine or azacitidine is administered subcutaneously and romidepsin is administered orally. In yet another embodiment, decitabine or azacitidine and romidepsin are administered orally.

In one embodiment, decitabine or azacitidine and romidepsin are administered intravenously, with administration of decitabine or azacitidine occurring 30 to 60 minutes prior to romidepsin, during a cycle of four to forty weeks. In another embodiment, decitabine or azacitidine is administered subcutaneously and romidepsin is administered by intravenous infusion. In another embodiment, decitabine or azacitidine is administered subcutaneously and romidepsin is administered orally. In yet another embodiment, decitabine or azacitidine and romidepsin are administered orally.

In one embodiment, decitabine or azacitidine and romidepsin are administered intravenously, simultaneously, during a cycle of four to forty weeks. In another embodiment, decitabine or azacitidine is administered subcutaneously and romidepsin is administered by intravenous infusion. In another embodiment, decitabine or azacitidine is administered subcutaneously and romidepsin is administered orally. In yet another embodiment, decitabine or azacitidine and romidepsin are administered orally.

In one embodiment, one cycle comprises the administration of from about 0.1 to about 4.0 mg per day of decitabine or azacitidine and from about 25 to about 150 mg/m² of romidepsin daily for three to four weeks and then one or two weeks of rest. In one embodiment, the number of cycles during which the combinatorial treatment is administered to a patient is from about one to about 40 cycles, or from about one to about 24 cycles, or from about two to about 16 cycles, or from about four to about three cycles.

In one embodiment, development of biomarkers predictive of maximal clinical benefit with romidepsin and decitabine or azacitidine would allow identification of those patients particularly suited for romidepsin and decitabine or azacitidine combination therapy. Thus, in one embodiment, provided herein are biomarkers that could be used, for example, in the management of therapeutic choices for patients with TNBC or ccRCC. In other embodiments, provided herein is a method of using a biomarker provided herein in selecting cancer patients for a particular therapy, e.g., romidepsin and decitabine or azacitidine combination therapy for a particular cancer, to derive maximal clinical benefits from that therapy.

In one embodiment, provided herein are predictive biomarkers for assessing potential clinical benefit of a cancer therapy. In one embodiment, provided herein are predictive biomarkers for assessing potential clinical benefit of romidepsin and decitabine or azacitidine combination therapy. In one embodiment, provided herein are methods of using a predictive biomarker provided herein (e.g., levels of expression of chromatin biomarkers) for assessing an efficacy of romidepsin and decitabine or azacitidine combination therapy. In one embodiment, the chromatin biomarkers provided herein include, but are not limited to, RhoB, p21, p15, p16, TβRIII, GATA3, sFRP1, sFRP2, sFRP4, sFRP5, DKK1 and DKK3. In a particular embodiment, the chromatin biomarker is sFRP1. In one embodiment, the biomarkers provided herein can be used to assess or predict response rate, overall survival, or other clinical benefits. In one embodiment, the clinical benefit includes, but is not limited to, prolonged survival, delayed progression to metastasis and/or other beneficial clinical responses.

In one embodiment, provided herein are biomarkers for assessing clinical benefit or predicting long-term clinical response, after the initiation of a romidepsin and decitabine or azacitidine combination therapy (e.g., assessing clinical benefit or potential long-term clinical response in a patient after or during treatment with romidepsin and decitabine or azacitidine combination therapy). In one embodiment, provided herein are methods of using a biomarker provided herein by measuring a level of expression of chromatin biomarkers. For example, the levels of expression of chromatin markers in post-treatment samples may be compared to baseline samples (e.g., after a treatment cycle of about 1, about 2, about 3, about 4, about 5, about 6, about 7, about 8, about 9, about 10, about 11, about 12, or greater than about 12 months; or after a treatment cycle of about 1, about 2, about 3, about 4, about 5, about 6, about 7, about 8, about 9, about 10, about 12, about 14, about 16, about 18, about 20, about 22, about 24, about 26, about 28, about 30, about 32, about 34, about 36, about 38, about 40, about 42, about 44, about 46, about 48, about 50, about 52, about 54, about 56, or greater than about 56 weeks). In one embodiment, the expression levels of particular chromatin markers are monitored periodically after the initiation of a romidepsin and decitabine or azacitidine combination therapy. In one embodiment, the clinical benefit includes, but is not limited to, prolonged survival, delayed progression to metastasis, and/or other beneficial clinical responses.

In one embodiment, provided herein are biomarkers that could be used to predict which cancer patients will have the most, or least, clinical benefit from a particular cancer therapy. In one embodiment, the methods or biomarkers provided herein may be applied to cancers, such as, solid cancers, or a type of cancer described herein elsewhere. See, e.g., International Patent Application No. PCT/US2010/000361, filed Feb. 9, 2010, published as WO2010/093435, incorporated herein by reference in its entirety. In one embodiment, the methods or biomarkers provided herein may be applied to TNBC or ccRCC. In one embodiment, the biomarkers provided herein are chromatin biomarkers selected from the group cocsisting of RhoB, p21, p15, p16, TβRIII, GATA3, sFRP1, sFRP2, sFRP4, sFRP5, DKK1 and DKK3. In a particular embodiment, the chromatin biomarker is sFRP1. In other embodiments, the levels of expression of the chromatin biomarkers may be used as a biomarker in a method described herein.

For example, bio-samples can be obtained from patients having a certain cancer (e.g., blood or tissue samples may be used in a method provided herein). In one embodiment, the expression levels of one or more chromatin markers are measured for a particular patient and compared with reference values. In one embodiment, patients are grouped or selected based on the expression levels of one or more chromatin markers. In one embodiment, selected patients are further treated with a particular therapy to derive maximal response or clinical benefit. In one embodiment, the particular therapy is a romidepsin and decitabine or azacitidine combination therapy. In one embodiment, a patient is having TNBC or ccRCC.

In one embodiment, bio-samples (e.g., blood or tissue samples) are obtained from patients pre-treatment (e.g., from TNBC or ccRCC patients before receiving certain treatment). In one embodiment, the level of expression of one or more chromatin markers provided herein is measured. In one embodiment, the level of expression of one or more chromatin markers of a patient is compared with reference value(s). In one embodiment, a particular level of expression of one or more chromatin markers is used to distinguish patients having potentially greater or lesser response to or overall survival benefit from a particular therapy (e.g., romidepsin and decitabine or azacitidine combination therapy). In one embodiment, a particular group of TNBC or ccRCC patients selected based on a method provided herein is treated with romidepsin and decitabine or azacitidine combination therapy.

Compositions

Romidepsin and decitabine or azacitidine can be used as compositions when combined with an acceptable carrier or excipient. Such compositions are useful in the methods provided herein.

Provided herein are pharmaceutical compositions comprising romidepsin as an active ingredient, including an enantiomer, a mixture of enantiomers, a mixture of two or more diastereomers, a tautomer, a mixture of two or more tautomers, or an isotopic variant thereof; or a pharmaceutically acceptable salt, solvate, hydrate, or prodrug in combination with a pharmaceutically acceptable vehicle, carrier, diluent, or excipient, or a mixture thereof.

Provided herein are pharmaceutical compositions comprising decitabine or azacitidine as an active ingredient or a pharmaceutically acceptable salt, solvate, hydrate, or prodrug in combination with a pharmaceutically acceptable vehicle, carrier, diluent, or excipient, or a mixture thereof.

Suitable excipients are well known to those skilled in the art, and non-limiting examples of suitable excipients are provided herein. Whether a particular excipient is suitable for incorporation into a pharmaceutical composition or dosage form depends on a variety of factors well known in the art, including, but not limited to, the method of administration. For example, oral dosage forms such as tablets may contain excipients not suited for use in parenteral dosage forms. The suitability of a particular excipient may also depend on the specific active ingredients in the dosage form. For example, the decomposition of some active ingredients may be accelerated by some excipients such as lactose, or when exposed to water. Active ingredients that comprise primary or secondary amines are particularly susceptible to such accelerated decomposition. Consequently, provided herein are pharmaceutical compositions and dosage forms that contain little, if any, lactose other mono- or disaccharides. As used herein, the term “lactose-free” means that the amount of lactose present, if any, is insufficient to substantially increase the degradation rate of an active ingredient. In one embodiment, lactose-free compositions comprise an active ingredient provided herein, a binder/filler, and a lubricant. In another embodiment, lactose-free dosage forms comprise an active ingredient, microcrystalline cellulose, pre-gelatinized starch, and magnesium stearate.

Like the amounts and types of excipients, the amounts and specific types of active ingredients in a dosage form may differ depending on factors such as, but not limited to, the route by which it is to be administered to patients. In one embodiment, dosage forms provided herein comprise romidepsin or a pharmaceutically acceptable salt, solvate, hydrate, stereoisomer, clathrate, or prodrug thereof in an amount of from about 0.5 mg/m² to 28 mg/m². In another embodiment, dosage forms provided herein comprise romidepsin or a pharmaceutically acceptable salt, solvate, hydrate, stereoisomer, clathrate, or prodrug thereof in an amount of about 8 mg/m², 10 mg/m², 12 mg/m², or 14 mg/m².

In one embodiment, dosage forms provided herein comprise decitabine or azacitidine or a pharmaceutically acceptable salt, solvate, hydrate, stereoisomer, clathrate, or prodrug thereof in an amount of from about 10 to about 150 mg/m². In another embodiment, dosage forms provided herein comprise decitabine or azacitidine or a pharmaceutically acceptable salt, solvate, hydrate, stereoisomer, clathrate, or prodrug thereof in an amount of about 10, 15, 25, 50, 75, 100, 125, or 150 mg/m². In a specific embodiment, a dosage form comprises decitabine or azacitidine in an amount of about 15, 25, 50, 75 or 100 mg/m².

Pharmaceutical compositions provided herein can be used in the preparation of individual, single unit dosage forms. Single unit dosage forms are suitable for oral, mucosal (e.g., nasal, sublingual, vaginal, buccal, or rectal), parenteral (e.g., subcutaneous, intravenous, bolus injection, intramuscular, or intraarterial), topical (e.g., eye drops or other ophthalmic preparations), transdermal or transcutaneous administration to a patient. Examples of dosage forms include, but are not limited to: tablets; caplets; capsules, such as soft elastic gelatin capsules; cachets; troches; lozenges; dispersions; suppositories; powders; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; eye drops or other ophthalmic preparations suitable for topical administration; and sterile solids (e.g., crystalline or amorphous solids) that can be reconstituted to provide liquid dosage forms suitable for parenteral administration to a patient.

In one embodiment, the pharmaceutical compositions provided herein formulated in various dosage forms for oral administration.

In one embodiment, the pharmaceutical compositions provided herein formulated in various dosage forms for parenteral administration. In a specific embodiment, the pharmaceutical compositions provided herein formulated in various dosage forms for intravenous administration. In a specific embodiment, the pharmaceutical compositions provided herein formulated in various dosage forms for subcutaneous administration.

In one embodiment, the pharmaceutical compositions are provided in a dosage form for oral administration, which comprise romidepsin or a pharmaceutically acceptable salt, solvate, hydrate, or prodrug thereof; and one or more pharmaceutically acceptable excipients or carriers. In one embodiment, a dosage form is a capsule or tablet comprising romidepsin in an amount of about 10 mg/m², 25 mg/m², 50 mg/m², 100 mg/m², 200 mg/m², or 300 mg/m². In another embodiment, capsule or tablet dosage form comprises romidepsin in an amount of about 50 mg/m² or 75 mg/m².

In one embodiment, the pharmaceutical compositions are provided in a dosage form for parenteral administration, which comprise romidepsin or a pharmaceutically acceptable salt, solvate, hydrate, or prodrug thereof; and one or more pharmaceutically acceptable excipients or carriers. In one embodiment, a dosage form is a syringe or vial comprising romidepsin in an amount of about 0.5 mg/m², 2.5 mg/m², 7.5 mg/m², 15 mg/m², 20 mg/m², or 28 mg/m². In another embodiment, syringe or vial dosage form comprises romidepsin in an amount of about 8 mg/m², 10 mg/m², 12 mg/m², or 14 mg/m².

In one embodiment, the pharmaceutical compositions are provided in a dosage form for parenteral administration, which comprise decitabine or azacitidine or a pharmaceutically acceptable salt, solvate, hydrate, or prodrug thereof; and one or more pharmaceutically acceptable excipients or carriers. In one embodiment, a dosage form is a syringe or vial comprising decitabine or azacitidine in the amount of 10, 15, 25, 50, 75, 100, 125, or 150 mg/m². In another embodiment, a syringe or vial dosage form comprises decitabine or azacitidine in an amount of about 10, 15, 25, 50, 75, or 100 mg/m².

The pharmaceutical compositions provided herein can be provided in a unit-dosage form or multiple-dosage form. Examples of a unit-dosage form include an ampoule, syringe, and individually packaged tablet and capsule. For example, a 100 mg unit dose contains about 100 mg of an active ingredient in a packaged tablet or capsule. A unit-dosage form may be administered in fractions or multiples thereof. A multiple-dosage form is a plurality of identical unit-dosage forms packaged in a single container to be administered in segregated unit-dosage form. Examples of a multiple-dosage form include a vial, bottle of tablets or capsules, or bottle of pints or gallons.

The pharmaceutical compositions provided herein can be administered at once, or multiple times at intervals of time. It is understood that the precise dosage and duration of treatment may vary with the age, weight, and condition of the patient being treated, and may be determined empirically using known testing protocols or by extrapolation from in vivo or in vitro test or diagnostic data. It is further understood that for any particular individual, specific dosage regimens should be adjusted over time according to the individual need and the professional judgment of the person administering or supervising the administration of the formulations.

### A. Oral Administration

The pharmaceutical compositions provided herein for oral administration can be provided in solid, semisolid, or liquid dosage forms for oral administration. As used herein, oral administration also includes buccal, lingual, and sublingual administration. Suitable oral dosage forms include, but are not limited to, tablets, fastmelts, chewable tablets, capsules, pills, strips, troches, lozenges, pastilles, cachets, pellets, medicated chewing gum, bulk powders, effervescent or non-effervescent powders or granules, oral mists, solutions, emulsions, suspensions, wafers, sprinkles, elixirs, and syrups. In addition to the active ingredient(s), the pharmaceutical compositions can contain one or more pharmaceutically acceptable carriers or excipients, including, but not limited to, binders, fillers, diluents, disintegrants, wetting agents, lubricants, glidants, coloring agents, dye-migration inhibitors, sweetening agents, flavoring agents, emulsifying agents, suspending and dispersing agents, preservatives, solvents, non-aqueous liquids, organic acids, and sources of carbon dioxide.

Binders or granulators impart cohesiveness to a tablet to ensure the tablet remaining intact after compression. Suitable binders or granulators include, but are not limited to, starches, such as corn starch, potato starch, and pre-gelatinized starch (e.g., STARCH 1500); gelatin; sugars, such as sucrose, glucose, dextrose, molasses, and lactose; natural and synthetic gums, such as acacia, alginic acid, alginates, extract of Irish moss, panwar gum, ghatti gum, mucilage of isabgol husks, carboxymethylcellulose, methylcellulose, polyvinylpyrrolidone (PVP), Veegum, larch arabogalactan, powdered tragacanth, and guar gum; celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); microcrystalline celluloses, such as AVICEL-PH-101, AVICEL-PH-103, AVICEL RC-581, AVICEL-PH-105 (FMC Corp., Marcus Hook, Pa.); and mixtures thereof. Suitable fillers include, but are not limited to, talc, calcium carbonate, microcrystalline cellulose, powdered cellulose, dextrates, kaolin, mannitol, silicic acid, sorbitol, starch, pre-gelatinized starch, and mixtures thereof. The amount of a binder or filler in the pharmaceutical compositions provided herein varies upon the type of formulation, and is readily discernible to those of ordinary skill in the art. The binder or filler may be present from about 50 to about 99% by weight in the pharmaceutical compositions provided herein.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets. The amount of a diluent in the pharmaceutical compositions provided herein varies upon the type of formulation, and is readily discernible to those of ordinary skill in the art.

Suitable disintegrants include, but are not limited to, agar; bentonite; celluloses, such as methylcellulose and carboxymethylcellulose; wood products; natural sponge; cation-exchange resins; alginic acid; gums, such as guar gum and Veegum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; aligns; and mixtures thereof. The amount of a disintegrant in the pharmaceutical compositions provided herein varies upon the type of formulation, and is readily discernible to those of ordinary skill in the art. The amount of a disintegrant in the pharmaceutical compositions provided herein varies upon the type of formulation, and is readily discernible to those of ordinary skill in the art. The pharmaceutical compositions provided herein may contain from about 0.5 to about 15% or from about 1 to about 5% by weight of a disintegrant.

Suitable lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; talc; hydrogenated vegetable oil, including peanut oil, cottonseed oil, sunflower oil, sesame oil, olive oil, corn oil, and soybean oil; zinc stearate; ethyl oleate; ethyl laureate; agar; starch; lycopodium; silica or silica gels, such as AEROSIL® 200 (W.R. Grace Co., Baltimore, Md.) and CAB-O-SIL® (Cabot Co. of Boston, Mass.); and mixtures thereof. The pharmaceutical compositions provided herein may contain about 0.1 to about 5% by weight of a lubricant.

Suitable glidants include, but are not limited to, colloidal silicon dioxide, CAB-O-SIL® (Cabot Co. of Boston, Mass.), and asbestos-free talc. Suitable coloring agents include, but are not limited to, any of the approved, certified, water soluble FD&C dyes, and water insoluble FD&C dyes suspended on alumina hydrate, and color lakes and mixtures thereof. A color lake is the combination by adsorption of a water-soluble dye to a hydrous oxide of a heavy metal, resulting in an insoluble form of the dye. Suitable flavoring agents include, but are not limited to, natural flavors extracted from plants, such as fruits, and synthetic blends of compounds which produce a pleasant taste sensation, such as peppermint and methyl salicylate. Suitable sweetening agents include, but are not limited to, sucrose, lactose, mannitol, syrups, glycerin, and artificial sweeteners, such as saccharin and aspartame. Suitable emulsifying agents include, but are not limited to, gelatin, acacia, tragacanth, bentonite, and surfactants, such as polyoxyethylene sorbitan monooleate (TWEEN® 20), polyoxyethylene sorbitan monooleate 80 (TWEEN® 80), and triethanolamine oleate. Suitable suspending and dispersing agents include, but are not limited to, sodium carboxymethylcellulose, pectin, tragacanth, Veegum, acacia, sodium carbomethylcellulose, hydroxypropyl methylcellulose, and polyvinylpyrrolidone.

Suitable preservatives include, but are not limited to, glycerin, methyl and propylparaben, benzoic add, sodium benzoate and alcohol. Suitable wetting agents include, but are not limited to, propylene glycol monostearate, sorbitan monooleate, diethylene glycol monolaurate, and polyoxyethylene lauryl ether. Suitable solvents include, but are not limited to, glycerin, sorbitol, ethyl alcohol, and syrup. Suitable non-aqueous liquids utilized in emulsions include, but are not limited to, mineral oil and cottonseed oil. Suitable organic acids include, but are not limited to, citric and tartaric acid. Suitable sources of carbon dioxide include, but are not limited to, sodium bicarbonate and sodium carbonate.

It should be understood that many carriers and excipients may serve a plurality of functions, even within the same formulation.

The pharmaceutical compositions provided herein for oral administration can be provided as compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenyl salicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets.

The tablet dosage forms can be prepared from the active ingredient in powdered, crystalline, or granular forms, alone or in combination with one or more carriers or excipients described herein, including binders, disintegrants, controlled-release polymers, lubricants, diluents, and/or colorants. Flavoring and sweetening agents are especially useful in the formation of chewable tablets and lozenges.

The pharmaceutical compositions provided herein for oral administration can be provided as soft or hard capsules, which can be made from gelatin, methylcellulose, starch, or calcium alginate. The hard gelatin capsule, also known as the dry-filled capsule (DFC), consists of two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl- and propyl-parabens, and sorbic acid. The liquid, semisolid, and solid dosage forms provided herein may be encapsulated in a capsule. Suitable liquid and semisolid dosage forms include solutions and suspensions in propylene carbonate, vegetable oils, or triglycerides. Capsules containing such solutions can be prepared as described in U.S. Pat. Nos. 4,328,245; 4,409,239; and 4,410,545. The capsules may also be coated as known by those of skill in the art in order to modify or sustain dissolution of the active ingredient.

The pharmaceutical compositions provided herein for oral administration can be provided in liquid and semisolid dosage forms, including emulsions, solutions, suspensions, elixirs, and syrups. An emulsion is a two-phase system, in which one liquid is dispersed in the form of small globules throughout another liquid, which can be oil-in-water or water-in-oil. Emulsions may include a pharmaceutically acceptable non-aqueous liquid or solvent, emulsifying agent, and preservative. Suspensions may include a pharmaceutically acceptable suspending agent and preservative. Aqueous alcoholic solutions may include a pharmaceutically acceptable acetal, such as a di(lower alkyl)acetal of a lower alkyl aldehyde, e.g., acetaldehyde diethyl acetal; and a water-miscible solvent having one or more hydroxyl groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

Other useful liquid and semisolid dosage forms include, but are not limited to, those containing the active ingredient(s) provided herein, and a dialkylated mono- or poly-alkylene glycol, including, 1,2-dimethoxymethane, diglyme, triglyme, tetraglyme, polyethylene glycol-350-dimethyl ether, polyethylene glycol-550-dimethyl ether, polyethylene glycol-750-dimethyl ether, wherein 350, 550, and 750 refer to the approximate average molecular weight of the polyethylene glycol. These formulations can further comprise one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, bisulfite, sodium metabisulfite, thiodipropionic acid and its esters, and dithiocarbamates.

The pharmaceutical compositions provided herein for oral administration can be also provided in the forms of liposomes, micelles, microspheres, or nanosystems. Micellar dosage forms can be prepared as described in U.S. Pat. No. 6,350,458.

The pharmaceutical compositions provided herein for oral administration can be provided as non-effervescent or effervescent, granules and powders, to be reconstituted into a liquid dosage form. Pharmaceutically acceptable carriers and excipients used in the non-effervescent granules or powders may include diluents, sweeteners, and wetting agents. Pharmaceutically acceptable carriers and excipients used in the effervescent granules or powders may include organic acids and a source of carbon dioxide.

Coloring and flavoring agents can be used in all of the above dosage forms.

The pharmaceutical compositions provided herein for oral administration can be formulated as immediate or modified release dosage forms, including delayed-, sustained, pulsed-, controlled, targeted-, and programmed-release forms.

### B. Parenteral Administration

The pharmaceutical compositions provided herein can be administered parenterally by injection, infusion, or implantation, for local or systemic administration. Parenteral administration, as used herein, include intravenous, intraarterial, intraperitoneal, intrathecal, intraventricular, intraurethral, intrasternal, intracranial, intramuscular, intrasynovial, intravesical, and subcutaneous administration.

The pharmaceutical compositions provided herein for parenteral administration can be formulated in any dosage forms that are suitable for parenteral administration, including solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science (see, Remington: The Science and Practice of Pharmacy, supra).

The pharmaceutical compositions intended for parenteral administration can include one or more pharmaceutically acceptable carriers and excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

Suitable aqueous vehicles include, but are not limited to, water, saline, physiological saline or phosphate buffered saline (PBS), sodium chloride injection, Ringers injection, isotonic dextrose injection, sterile water injection, dextrose and lactated Ringers injection. Suitable non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, and palm seed oil. Suitable water-miscible vehicles include, but are not limited to, ethanol, 1,3-butanediol, liquid polyethylene glycol (e.g., polyethylene glycol 300 and polyethylene glycol 400), propylene glycol, glycerin, N-methyl-2-pyrrolidone, N,N-dimethylacetamide, and dimethyl sulfoxide.

Suitable antimicrobial agents or preservatives include, but are not limited to, phenols, cresols, mercurials, benzyl alcohol, chlorobutanol, methyl and propyl p-hydroxybenzoates, thimerosal, benzalkonium chloride (e.g., benzethonium chloride), methyl- and propyl-parabens, and sorbic acid. Suitable isotonic agents include, but are not limited to, sodium chloride, glycerin, and dextrose. Suitable buffering agents include, but are not limited to, phosphate and citrate. Suitable antioxidants are those as described herein, including bisulfite and sodium metabisulfite. Suitable local anesthetics include, but are not limited to, procaine hydrochloride. Suitable suspending and dispersing agents are those as described herein, including sodium carboxymethylcellulose, hydroxypropyl methylcellulose, and polyvinylpyrrolidone. Suitable emulsifying agents are those described herein, including polyoxyethylene sorbitan monolaurate, polyoxyethylene sorbitan monooleate 80, and triethanolamine oleate. Suitable sequestering or chelating agents include, but are not limited to EDTA. Suitable pH adjusting agents include, but are not limited to, sodium hydroxide, hydrochloric acid, citric acid, and lactic acid. Suitable complexing agents include, but are not limited to, cyclodextrins, including α-cyclodextrin, β-cyclodextrin, hydroxypropyl-β-cyclodextrin, sulfobutylether-β-cyclodextrin, and sulfobutylether 7-β-cyclodextrin (CAPTISOL®, CyDex, Lenexa, Kans.).

When the pharmaceutical compositions provided herein are formulated for multiple dosage administration, the multiple dosage parenteral formulations must contain an antimicrobial agent at bacteriostatic or fungistatic concentrations. All parenteral formulations must be sterile, as known and practiced in the art.

In one embodiment, the pharmaceutical compositions for parenteral administration are provided as ready-to-use sterile solutions. In another embodiment, the pharmaceutical compositions are provided as sterile dry soluble products, including lyophilized powders and hypodermic tablets, to be reconstituted with a vehicle prior to use. In yet another embodiment, the pharmaceutical compositions are provided as ready-to-use sterile suspensions. In yet another embodiment, the pharmaceutical compositions are provided as sterile dry insoluble products to be reconstituted with a vehicle prior to use. In still another embodiment, the pharmaceutical compositions are provided as ready-to-use sterile emulsions.

The pharmaceutical compositions provided herein for parenteral administration can be formulated as immediate or modified release dosage forms, including delayed-, sustained, pulsed-, controlled, targeted-, and programmed-release forms.

The pharmaceutical compositions provided herein for parenteral administration can be formulated as a suspension, solid, semi-solid, or thixotropic liquid, for administration as an implanted depot. In one embodiment, the pharmaceutical compositions provided herein are dispersed in a solid inner matrix, which is surrounded by an outer polymeric membrane that is insoluble in body fluids but allows the active ingredient in the pharmaceutical compositions diffuse through.

Suitable inner matrixes include, but are not limited to, polymethylmethacrylate, polybutyl-methacrylate, plasticized or unplasticized polyvinylchloride, plasticized nylon, plasticized polyethylene terephthalate, natural rubber, polyisoprene, polyisobutylene, polybutadiene, polyethylene, ethylene-vinyl acetate copolymers, silicone rubbers, polydimethylsiloxanes, silicone carbonate copolymers, hydrophilic polymers, such as hydrogels of esters of acrylic and methacrylic acid, collagen, cross-linked polyvinyl alcohol, and cross-linked partially hydrolyzed polyvinyl acetate.

Suitable outer polymeric membranes include but are not limited to, polyethylene, polypropylene, ethylene/propylene copolymers, ethylene/ethyl acrylate copolymers, ethylene/vinyl acetate copolymers, silicone rubbers, polydimethyl siloxanes, neoprene rubber, chlorinated polyethylene, polyvinylchloride, vinyl chloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer.

### C. Delayed Release Dosage Forms

Pharmaceutical compositions comprising romidepsin and 3-(4-amino-1-oxo-1,3-dihydro-isoindol-2-yl)-piperidine-2,6-dione can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled-release.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

Romidepsin Formulation

In one embodiment, romidepsin is formulated for injection as a sterile lyophilized white powder and is supplied in a single-use vial containing 10 mg romidepsin and 20 mg povidone, USP. The diluent is a sterile clear solution and is supplied in a single-use vial containing a 2 ml deliverable volume. The diluent for romidepsin contains 80% (v/v) propylene glycol, USP and 20% (v/v) dehydrated alcohol, USP. Romidepsin is supplied as a kit containing two vials.

Romidepsin for injection is intended for intravenous infusion after reconstitution with the supplied Diluent and after further dilution with 0.9% Sodium Chloride, USP.

Azacitidine Formulation

In one embodiment, azacitidine is formulated for injection as a sterile lyophilized powder and is supplied in a single-use vial containing 100 mg of azacitidine and 100 mg of mannitol.

Azacitidine for injection is intended for intravenous injection after reconstitution as a solution with further dilution. Azacitidine for injection is intended for subcutaneous injection after reconstitution as a suspension.

Decitabine Formulation

In one embodiment, decitabine is formulated for injection as a white to almost white sterile lyophilized powder that is supplied in a clear colorless glass vial. Each vial (a single dose of 20 mL) contains 50 mg of decitabine, 68 mg of monobasic potassium phosphate (potassium dihydrogen phosphate) and 11.6 mg of sodium hydroxide.

Kits

In one embodiment, provided herein are kits comprising one or more containers filled with romidepsin or a pharmaceutical composition thereof, and one or more containers filled with azacitidine or decitabine or a pharmaceutical composition thereof.

### EXAMPLES

The following examples are provided by way of illustration, not limitation.

### Example 1

### Cell Line Verification

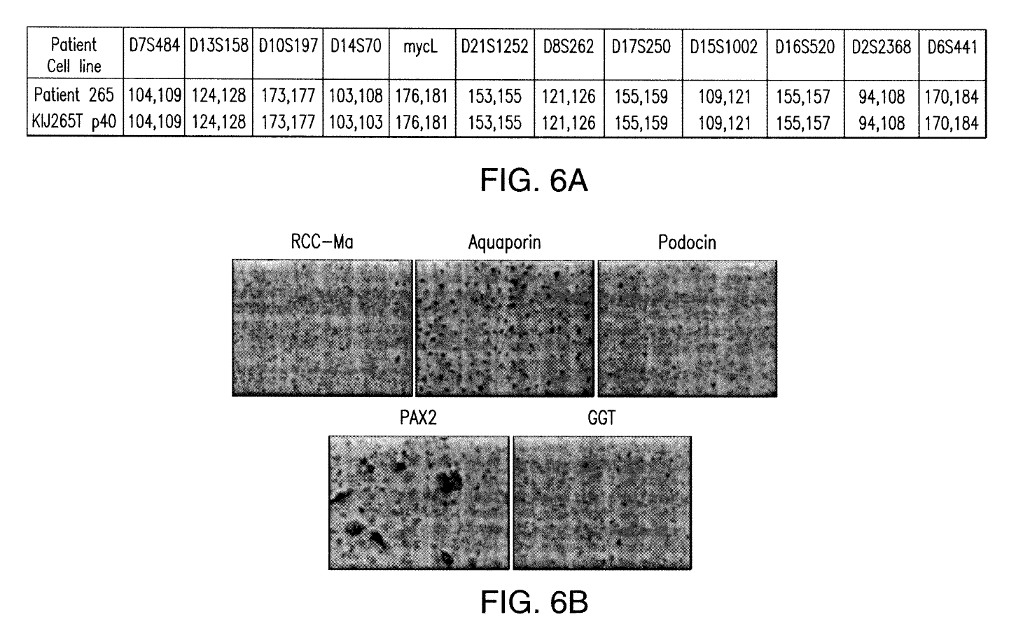

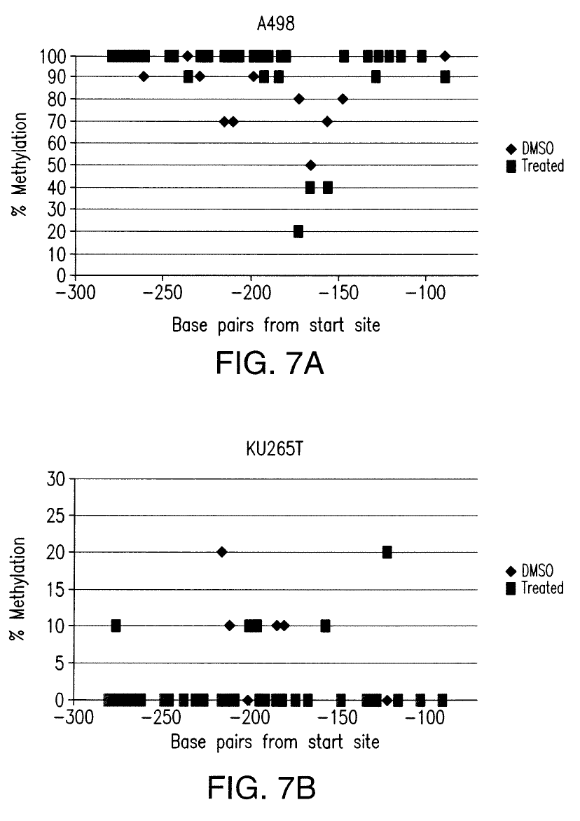

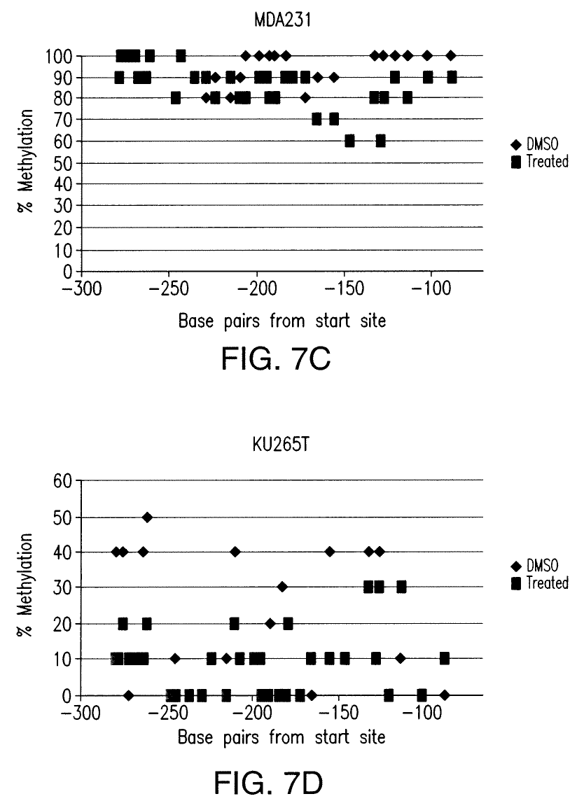

STR analysis was performed by Mayo Clinic Rochester core facility for KIJ265T samples. Genomic DNA from primary tissues and matching cell lines was isolated using the Purelink™ Genomic DNA mini kit (Invitrogen). Twelve renal specific STR markers were amplified in PCR reactions using fluorescently labeled primers from ABI (Applied Biosystems). Results were analyzed using ABI 3130 (Applied Biosystems). Markers included: D7S484, D13S158, D10S197, D14S70, mycL, D21S1252, D8S262, D17S250, D15S1002, D16S520, D2S2368, and D6S441. Peak sizes were calculated versus a co-injected size standard using Gene Marker (Soft Genetics, State College, Pa.). Immunohistochemistry was used to validate the renal origin of KIJ265T cells (FIGS. 6A and 6B). Cells were plated on slides, fixed using 2% paraformaldehyde (Sigma), permeabilized using 1% Triton X-100 (Sigma), and then blocked with diluent containing background reducing components (Dakocytomation, Denmark) prior to staining with primary antibody. Control slides were prepared by excluding primary antibody during staining. Primary antibodies included RCC-Ma (Cell Marque Corporation, Rocklin, Calif.), podocin (ABCAM, Cambridge, Mass.), gamma glutamyl transpeptidase (Lifespan Biosciences, Seattle, Wash.), PAX2 (Lifespan), and aquaporin2 (Santa Cruz, Santa Cruz, Calif.). The KIJ265T cell line was identified to be VHL mutant (Exon 2 c.407T>C; protein modification of p.F136S) by DNA sequencing.

### Example 2

### Cell Culture

Human renal cell carcinoma cell lines A498 (ATCC, Manassas, Va.) and KIJ265T (derived from primary tumor site stage 4 human renal cell carcinoma patient tissue established in the Copland laboratory), as well as triple negative breast cancer cell lines BT-20 (ATCC) and MDA-231 (ATCC) were maintained in phenol red free DMEM medium (Cellgro, Herndon, Va.) supplemented with 10% FBS (Hyclone, Logan, Utah) and penicillin-streptomycin (Invitrogen, Carlsbad, Calif.) at 37° C. in humidified conditions with 5% CO₂.

### Example 3

### Drug Treatments and Proliferation Assays

All drug stocks were prepared at 1000× concentration in DMSO. Cells were plated (1×10⁵ per well) per ml of media in 12-well plates (Midwest Scientific, St. Louis, Mo.) and each treatment carried out in triplicate. For mono-therapeutic treatments, cells were treated with a dose range of 0.01 μM to 10 μM for decitabine (purchased from Sigma-Aldrich, St. Louis, Mo.) or a dose range of 0.01 nM to 100 nM for romidepsin (provided by Gloucester Pharmaceuticals, Inc). DMSO was used for vehicle control. Cells were trypsinized 72 hours post treatment and counted using a Coulter Particle Counter (Beckman, Brea, Calif.). For combination dose-outs, cells were treated with a dose range of 0.1, 1, or 10 μM of decitabine. After 48 hours cells were treated with a dose range of 0.5 nM to 7.5 nM romidepsin for 24 hours. Cells were trypsinized 24 hours later and counted. Appropriate mono-therapeutic and DMSO treatments were included as control groups and administered in accordance with combinatorial time-points. An optimal combinatorial dose of 1 μM decitabine for 72 hours with the addition of 5 nM romidepsin for the last 24 hours was utilized in further treatments.

For the treatment of cells with recombinant human sFRP1, MDA231 and KIJ265T cells were seeded in a 96-well culture plate at 5000 cells per well in 100 μl DMEM supplemented with PSA and 10% FBS. After overnight incubation, cells were washed in PBS and 100 μl of serum free DMEM containing the final concentration of sFRP1 was added. The cells were incubated for 6 hours Serum was re-introduced to these wells to a final concentration of 2% FBS. Plates were incubated for 72 hours before cells were trypsinized and counted.

### Example 4

### RNA Isolation, RT-PCR, and Quantitative PCR

RNA was isolated from each sample group and purified using the RNAqueous Midi Kit (Ambion, Austin, Tex.). The O.D. 260/280 mRNA ratio was between 1.8 and 2.0 for all samples and the 18S/28S bands were verified for purity on a 1% agarose gel. RNA was reverse transcribed using the High Capacity cDNA Reverse Transcriptase Kit (Applied Biosystems, Foster City, Calif.) per manufacturer's instruction. cDNA samples were combined with TaqMan® Fast Universal PCR Master Mix (Applied Biosystems) and TaqMan® FAM™ dye-labeled probes including sFRP1 (Hs00610060_m1), RhoB (Hs00269660_s1), p21 (Hs00355782_m1), (Hs00234257_m1), GATA3 (Hs00231122_m1), and GAPDH (Hs99999905_m1). Gene expression was analyzed using quantitative real-time PCR (QPCR). GAPDH was used as the normalization control. Fold change values between drug treated samples were compared to fold change values of DMSO control samples by the comparative C(T) method (Schmittgen et al., Nat Protocol 3:1101-1108, 2008).

### Example 5

### Protein Expression Analysis

Cells were seeded (1×107) on 100 mm plates (Midwest Scientific) and were treated with the optimal combinatorial dose determined in the treatment protocol. Cells were collected and lysed using M-PER reagent (Pierce, Rockford, Ill.) containing protease inhibitor cocktail (Roche, Mannheim, Germany) and phosphatase inhibitor (Pierce). Quantification and transfer to 0.2 μM Immobilon Psq membranes were carried out as described in Copland et al., Oncogene 25:2304-2317, 2006. Membranes were blocked using 5% milk in Tris-Buffered Saline plus Tween-20 (TBS-T) (Fisher Scientific, Fairlawn, N.J.) and incubated overnight with primary antibody [PARP (Cell Signaling, Boston, Mass.), caspase-3 (Cell Signaling), sFRP1 (Cell Signaling), or β-actin (Sigma-Aldrich)] at 4° C. Membranes were incubated for 1 hour in secondary species-specific horseradish peroxidase-labeled antibodies (Jackson Immunoresearch, West Grove, Pa.) diluted in 5% Milk TBS-T at room temperature. Supersignal chemiluminescent kit (Pierce) was used for detection.

### Example 6

### Cell Death Analysis Via Flow Cytometry

Cells were treated with the optimal combinatorial therapy and appropriate control groups were prepared. After 72 hour treatment both adhered and floating cells were collected, centrifuged at 4° C., and washed using DPBS. Cells were centrifuged and re-suspended in 1× cold binding buffer (BD Pharmingen, San Jose, Calif.) at a concentration of 1×10⁶ cells per ml. Cells were stained for 10 minutes with propidium iodide (BD Pharmingen), and FACS analysis was performed using Accuri C6 flow cytometer (Accuri, Ann Arbor, Mich.). Unstained cells were used as controls for setting the cell population parameters.

### Example 7

### Lentivirus and Infections

MISSION shRNA pLKO.1 constructs (Sigma-Aldrich) were used to make self-inactivating shRNA lentiviruses for sFRP1 [target sequence 5′-CGAGATGCTTAAGTGTGACAA-3′ (clone NM_{—}003012.3-758s1c1) (Sigma-Aldrich)], and a non-target random scrambled sequence (SHC002) which was used as the control. Lentiviruses were packaged using HEK293FT (ATCC®, Manassas, Va.) cells via transient transfection using Lipofectamine 2000 (Invitrogen) paired with ViraPower (Invitrogen). Supernatant containing virus was harvested 72 hours post-transfection and filtered using a 0.45 μm PVDF syringe filter (Millipore). For virus transduction MDA-231 or KIJ265T cells were seeded at 2×10⁷ cells per 100 mm plate in 5 mL of growth medium and were incubated with lentivirus plus 5 μg/ml polybrene (American Bioanalytical, Natick, Mass.) for 24 hours. Clones were identified by puromycin (Fisher Scientific) selection.

### Example 8

### Statistical Analysis

Data are presented as the mean±SD and comparisons of treatment groups were analyzed by 2-tailed paired Student's t test. Data for comparison of multiple groups are presented as mean±SD and were analyzed by ANOVA. p<0.05 was considered statistically significant.

### Example 9

### Effect of Single Drug Therapy on Cell Proliferation

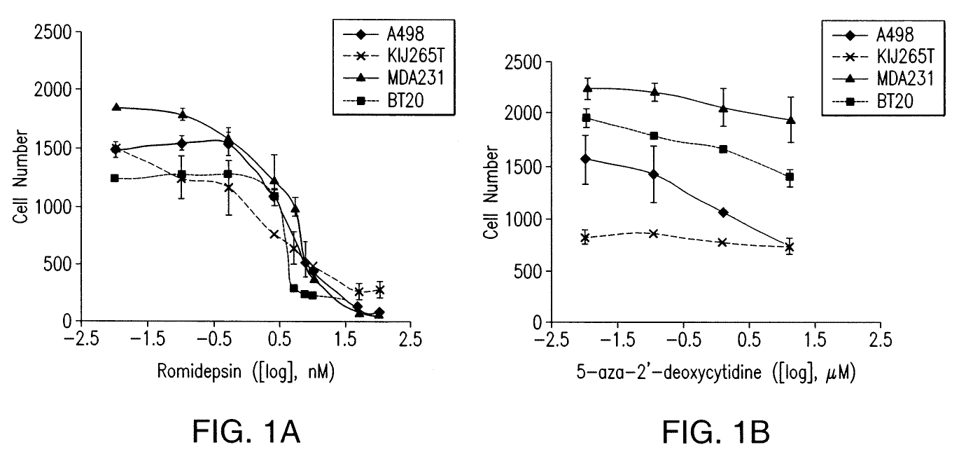

Individual drug treatments of romidepsin and decitabine were evaluated in two ccRCC stage 4 cell lines (A498 and KIJ265T) and two TNBC cell lines (MDA231 and BT20) for their ability to inhibit cell proliferation 72 hours post drug exposure (FIGS. 1A and 1B). Romidepsin produced significant inhibition of cell proliferation in the 2.5-100 nM dose range (FIG. 1A). Treatment with decitabine had minimal effect on cell proliferation at all tested doses, as shown in FIG. 1B. These data identify romidepsin as a potent inhibitor of cell proliferation in ccRCC and TNBC cell lines.

### Example 10

### Effect of Combinational Drug Therapy on Cell Proliferation

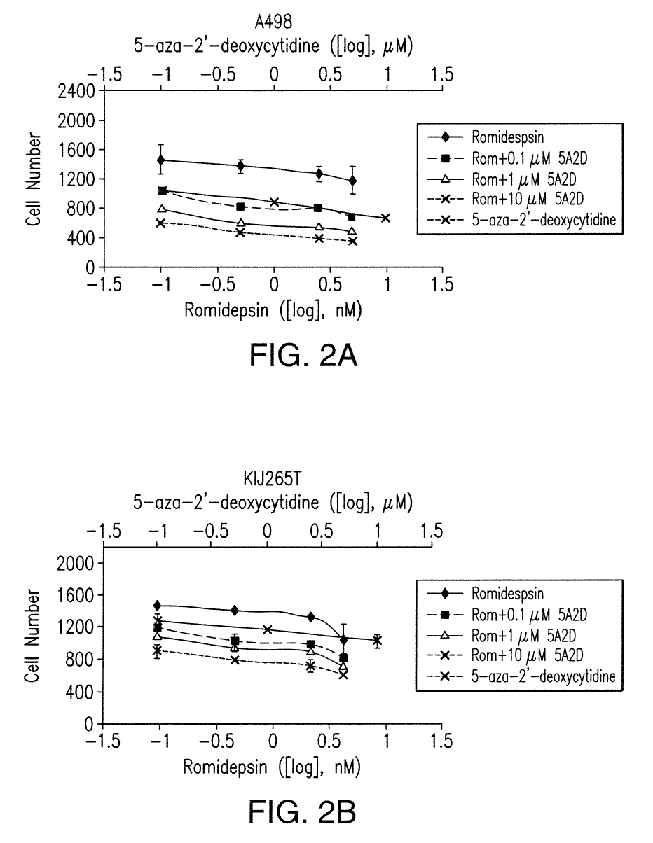

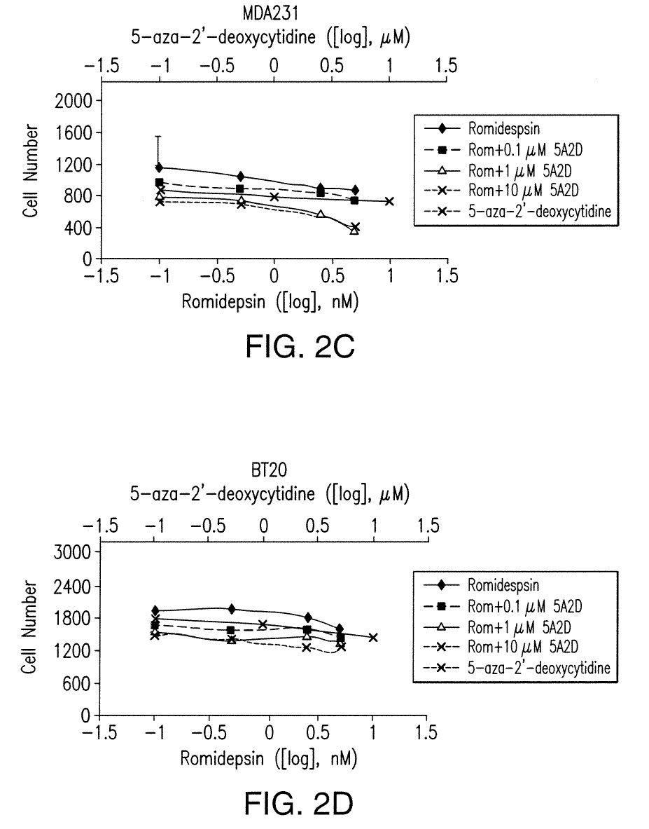

Combinatorial treatments of romidepsin and decitabine were evaluated for their ability to inhibit cell proliferation in ccRCC and TNBC cell lines (FIGS. 2A-2D). Romidepsin doses were standardized to 0.1, 0.5, 2.5 and 5 nM, with 5 nM being a dose that induced ˜50% cell death in single treatment samples over a 3 day exposure. Cells were treated with doses of decitabine at 0.1, 1 and 10 μM. Treatment protocols for these studies evaluated the response of the cells to either a 24 hour treatment of romidepsin alone, a 72 hour treatment of decitabine alone or treatment for 72 hours of decitabine with the final 24 hours in combination with romidepsin. In ccRCC and TNBC cell lines combinational drug treatment with romidepsin and decitabine induced greater inhibition of cell proliferation than single drug treatments alone.

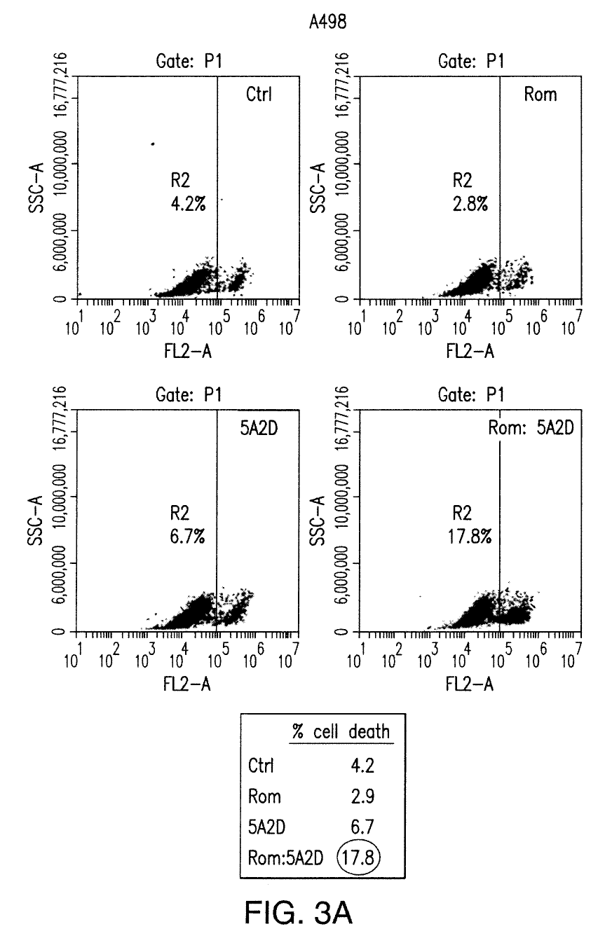

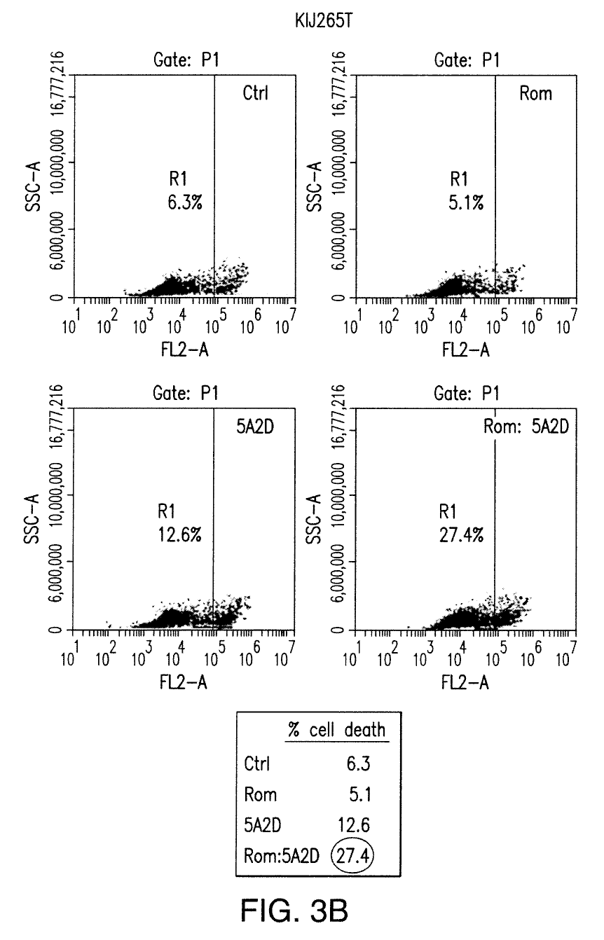

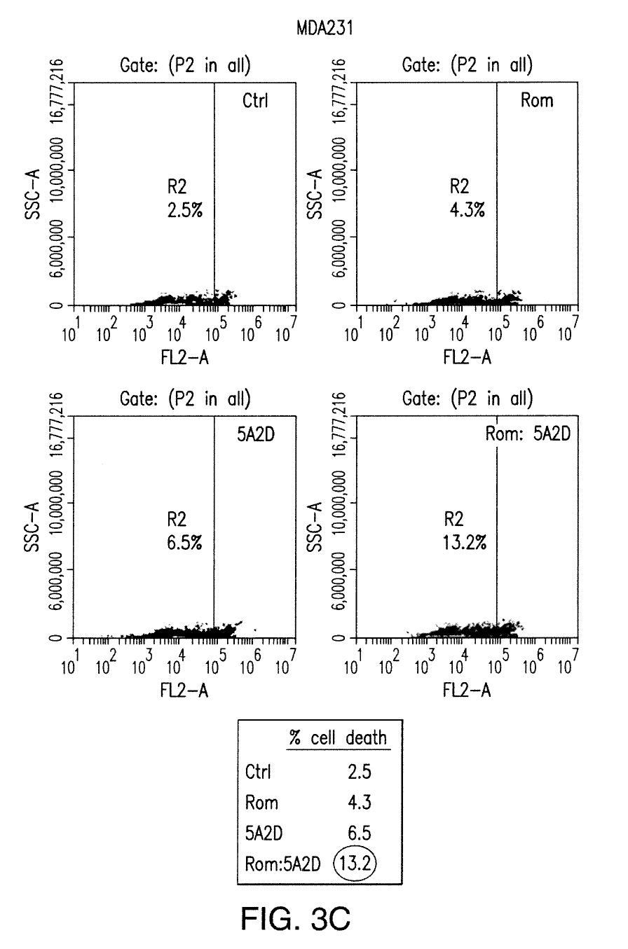

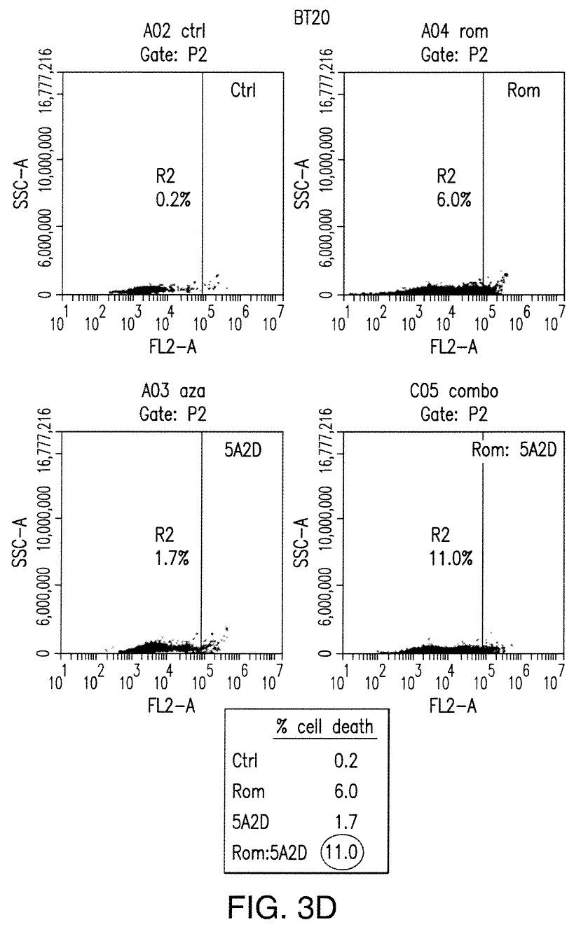

The cell lines were also analyzed for drug induced cell death. Propidium iodide staining of cell lines treated with 5 nM romidepsin or 1 μM decitabine alone or in combination identified a synergistic induction of cell death in the combination drug therapy group (FIGS. 3A and 3B). The greatest induction of cell death with combination treatment was observed in the ccRCC cell line KIJ265T with death being induced 21.1% above DMSO treated controls (FIG. 3B). For cell lines A498, MDA231 and BT20, cell death in the combination treatment group was induced when compared to DMSO controls by 13.6%, 10.7% and 10.8% respectively. Single drug exposures were unable to consistently induce cell death across the tested cell lines, although decitabine alone in KIJ265T and romidepsin in BT20 statistically increased mortality (6.3% and 5.8% above controls respectively).

### Example 11

### Effect of Combinational Drug Therapy on Apoptosis

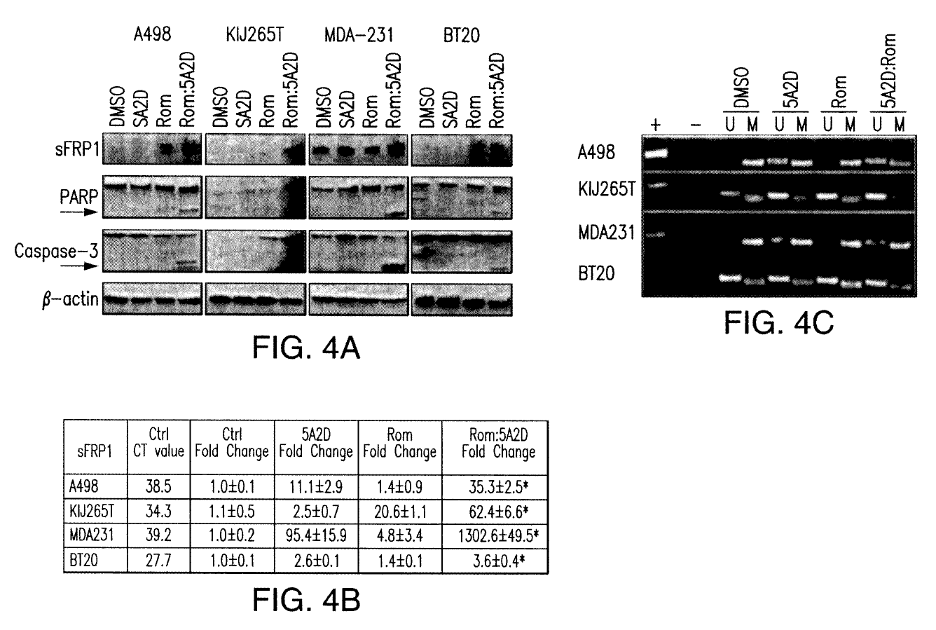

To investigate the mechanism of cell death in single and combination drug treated cells, total cellular proteins were analyzed for markers of apoptosis. Total protein from cells treated under the optimal dosing regime described above were harvested and examined by western blotting techniques. In all experimental cell lines, cleavage of both caspase-3 and PARP were absent in control or single drug treatment groups. However, treatment of cells with 5 nM romidepsin and 1 μM decitabine in combination caused cleavage of caspase-3 and PARP in all cell lines (FIG. 4A) indicating that in combination these drugs are potent inducers of apoptosis.

### Example 12

### Effect of Combinational Drug Therapy on Expression of sFRP1

To elucidate the molecular events taking place with the exposure of ccRCC and TNBC cells to romidepsin and decitabine in combination, we analyzed molecular targets that we have previously identified to be directly or indirectly affected by epigenetic silencing in cancer. Expression levels of protein and RNA from all experimental cell lines were examined for RhoB (Marlow et al., J Clin Endocrinol Metab 95:5337-5347, 2010; Marlow et al., Cancer Res 69:1536-1544, 2009) p21 (Marlow, supra), TβRIII (Cooper et al., Oncogene 29:2905-2915, 2010), GATA3 (Cooper, supra), and sFRP1 (Gumz, supra) after single or combinational treatments. Of the molecular targets investigated, sFRP1 showed consistent up-regulation of RNA expression across all cell lines treated with combinatorial therapy and up-regulation was confirmed at the protein level (FIGS. 4A and 4B). Thus, accumulation of sFRP1 expression was observed with combination treatment both at the protein level and RNA level as shown by real-time PCR. The fold change expression values of sFRP1 in treated samples were normalized to DMSO controls for each cell line identifying synergistic apoptotic escalation with combinatorial treatment. sFRP1 has been demonstrated to behave as a tumor suppressor gene. The re-expression of sFRP1 in dual therapy treated cancer cells is sufficient to modulate cell survival.

### Example 13

### Silencing of Dual Treatment Induced sFRP1 Leads to Gain of Cell Survival

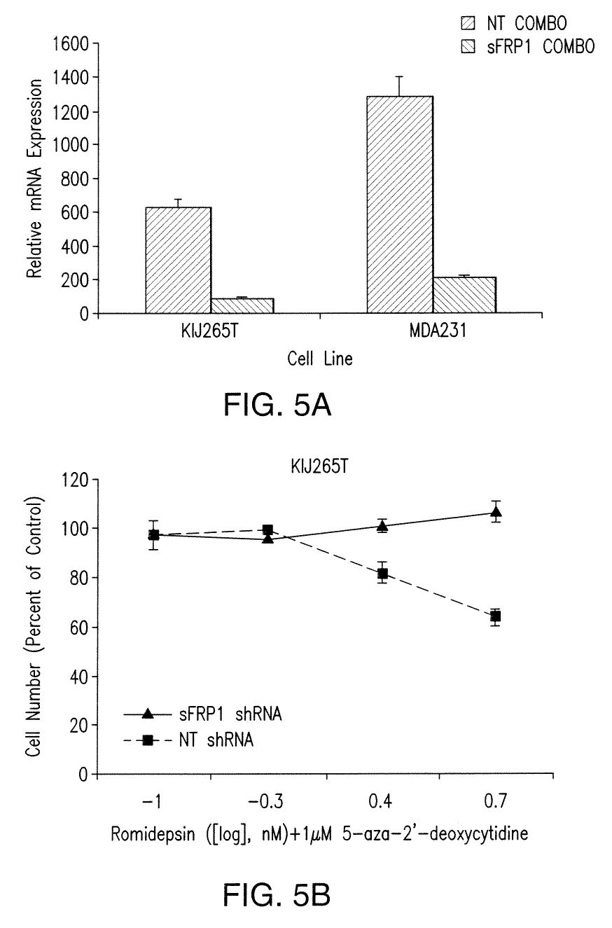

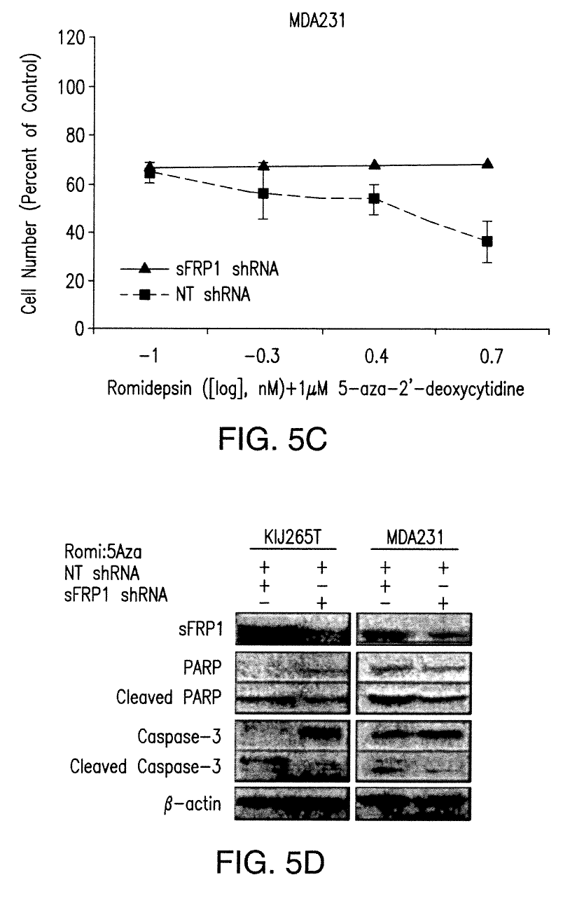

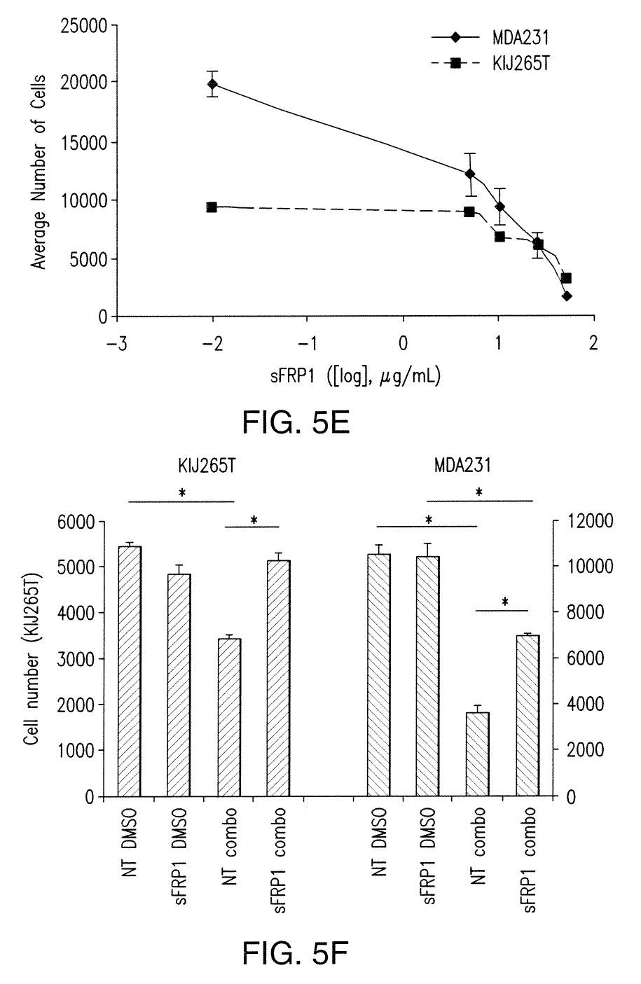

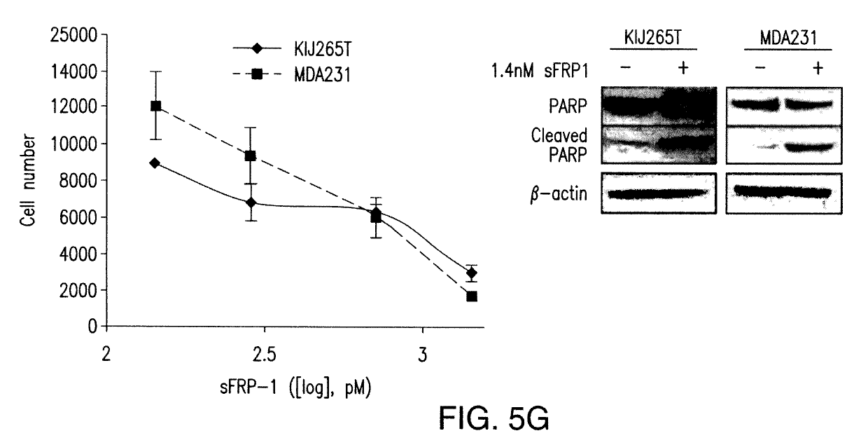

The endogenous levels of sFRP1 were silenced in TNBC (MDA231) and ccRCC (KIJ265T) cell lines using shRNA technologies. sFRP1 silenced cells were exposed to a combination of 5 nM romidepsin and 1 μM decitabine, and the expression of sFRP1 RNA message was evaluated (FIG. 5A). Combinatorial treatment induced expression of sFRP1 in the MDA231 and KIJ265T non-target cell lines ˜1300 and ˜600 fold, respectively, when compared to non-treated, non-target controls. With shRNA silencing of sFRP1, induction of RNA message with combinatorial treatment reduced this expression by 6 fold or greater in MDA231 and KIJ265T cells. The loss of inducible sFRP1 was observed to reduce the effects of combinatorial treatment in both KIJ265T and MDA231 cells. Following treatment with romidepsin and decitabine, the growth of sFRP1 shRNA silenced cells was minimally affected when compared to treated shRNA non-target controls. At the dose of romidepsin 5 nM and decitabine sFRP1 shRNA silenced cells were 1.7 fold and 1.8 fold less responsive to this combination than non-target treated controls for KIJ265T and MDA231 cells respectively (FIGS. 5B and 5C). Analysis of total cell proteins identified that sFRP1 shRNA silenced cells had reduced levels of PARP and caspase-3 cleaved products identifying that cell survival after combinatorial treatment in these cells was due to an inhibited apoptotic response (FIG. 5D). These data identify that sFRP1 is a target of romidepsin/decitabine treatment and that its re-expression plays a role in the inhibition of cell growth and induction of cell death.

### Example 14

### Effect of Recombinant sFRP1 on Cell Proliferation

To verify that epigenetic silencing of sFRP1 is vital for TNBC and ccRCC cell survival, we re-introduced recombinant human sFRP1 to the media of cells and examined its effect on cell proliferation. Escalating doses of recombinant sFRP1 in the media led to a dose-dependent decrease in MDA231 and KIJ265T cell proliferation (FIG. 5E) showing that the re-expression of sFRP1 is capable of inhibiting cancer cell growth. Therefore, romidepsin and decitabine in combination are a potential drug therapy option for the treatment of ccRCC and TNBC via synergistic re-expression of sFRP1 by the combination of these drugs.

Treatment of primary site, metastatic ccRCC and TNBC cell lines with combination therapy of romidepsin and decitabine showed synergistic inhibition of cell proliferation and induction of cell death via apoptosis. The combination of drugs caused re-expression of the tumor suppressor gene sFRP1, silencing of which plays a prominent role in survival of ccRCC and TNBC cells. Together these data suggest that romidepsin and decitabine in combination is a promising therapeutic drug regimen for the treatment of ccRCC and TNBC.

All publications, patents, and patent applications mentioned in this specification are herein incorporated by reference to the same extent as if each individual publication, patent, or patent application was specifically and individually indicated to be incorporated by reference.

The present disclosure has been described above with reference to exemplary embodiments. However, those skilled in the art, having read this disclosure, will recognize that changes and modifications may be made to the exemplary embodiments without departing from the scope of the present disclosure. The changes or modifications are intended to be included within the scope of the present disclosure, as expressed in the following claims.

What is claimed:

1. A method of treating a chemoresistant cancer, said method comprising administering to a patient in need of such treatment a therapeutically effective amount of an HDAC inhibitor and a therapeutically effective amount of a DNA demethylating agent.

2. The method of claim 1, wherein the chemoresistant cancer is triple negative breast cancer (TNBC).

3. The method of claim 1, wherein the chemoresistant cancer is clear cell renal cell carcinoma (ccRCC).

4. The method of claim 1, wherein the HDAC inhibitor is romidepsin.

5. The method of claim 1, wherein the DNA demethylating agent is a cytidine analog.

6. The method of claim 5, wherein the cytidine analog is azacitidine or decitabine.

7. The method of claim 1, wherein the HDAC inhibitor is romidepsin in an amount from about 0.5 to about 28 mg/m² per day and the DNA demethylating agent is a cytidine analog in an amount of about 10 to 150 mg/m² per day.

8. The method of claim 7, wherein cytidine analog and romidepsin are administered intravenously.

9. The method of claim 7, wherein the amount of the cytidine analog is about 15, 25, 50, 75 or 100 mg/m² per day.

10. The method of claim 7, wherein the amount of romidepsin is about 8, 10, 12 or 14 mg/m² per day.

11. The method of claim 1, wherein the HDAC inhibitor is romidepsin in an amount from about 10 to about 300 mg/m² per day and the DNA demethylating agent is a cytidine analog in an amount from about 10 to 150 mg/m² per day.

12. The method of claim 11, wherein the cytidine analog is administered intravenously and romidepsin is administered orally.

13. The method of claim 11, wherein the cytidine analog is administered subcutaneously and romidepsin is administered orally.

14. The method of claim 11, wherein the cytidine analog is administered orally and romidepsin is administered intravenously.

15. The method of claim 11, wherein the cytidine analog and romidepsin are administered orally.

16. The method of claim 11, wherein the amount of romidepsin is about from 25 to about 200 mg/m² per day.

17. The method of claim 11, wherein the amount of romidepsin administered is about 50, 75 or 100 mg/m² per day.

18. The method of claim 11, wherein the amount of the cytidine analog is about 15, 25, 50, 75 or 100 mg/m² per day.

19. The method of claim 1, wherein the demethylating agent is a cytidine analog administered in an amount of about 15, 25 50, 75 or 100 mg/m² per day for about 3 to about 14 days followed by about 21 to about 25 days rest in a 28 day cycle, and wherein the HDAC inhibitor is romidepsin administered in an amount of about 10 or 12 mg/m² per day on days 1, 8 and 15 of the 28 day cycle.

20. The method of claim 5, wherein the cytidine analog is decitabine.

21. The method of claim 5, wherein the cytidine analog is azacitidine.

22. A pharmaceutical composition comprising romidepsin and a cytidine analog.

23. The pharmaceutical composition of claim 22, wherein the cytidine analog is decitabine

24. The pharmaceutical composition of claim 22, wherein the cytidine analog is azacitidine.

25. A method for identifying a patient diagnosed with TNBC or ccRCC having an increased probability of obtaining improved overall survival following a combined treatment with romidepsin and a cytidine analog.

26. The method of claim 25, wherein the cytidine analog is decitabine.

27. The method of claim 25, wherein the cytidine analog is azacitidine.
